(19) United States
(12) Patent Application Publication
(10) Pub. No.: US 2025/0101115 A1
(43) Pub. Date: Mar. 27, 2025

(54) ANTI-MET FAB-FC FOR THE TREATMENT OF A TUMOR AND/OR METASTASIS

(71) Applicant: PIERRE FABRE MEDICAMENT, Lavaur (FR)

(72) Inventors: Elisa VIGNA, Villarbasse (Torino) (IT); Cristina BASILICO, Pavarolo (Torino) (IT); Tiziana CREPALDI, Torino (IT); Paolo COMOGLIO, Arignano (Torino) (IT)

(73) Assignee: PIERRE FABRE MEDICAMENT, Lavaur (FR)

(21) Appl. No.: 18/973,463

(22) Filed: Dec. 9, 2024

Related U.S. Application Data
(63) Continuation of application No. 17/283,482, filed on Apr. 7, 2021, now Pat. No. 12,209,129.

(30) Foreign Application Priority Data
Oct. 9, 2018 (IT) 102018000009282

(51) Int. Cl. C07K 16/28; A61K 39/00; A61P 35/00; A61P 35/04; C07K 16/46

(52) U.S. Cl. CPC C07K 16/2863; A61P 35/00; A61P 35/04; C07K 16/462; A61K 2039/505; C07K 2317/14; C07K 2317/21; C07K 2317/24; C07K 2317/33; C07K 2317/522; C07K 2317/524; C07K 2317/526; C07K 2317/53; C07K 2317/55; C07K 2317/565; C07K 2317/73; C07K 2317/732; C07K 2317/734; C07K 2317/76; C07K 2317/92

(57) ABSTRACT

An anti-Met antibody fragment comprising a single antigen binding arm and an Fc region, wherein the antigen binding arm is defined by the variable regions having amino acid sequences as set forth in SEQ ID No.: 7, 8 and wherein the anti-Met antibody fragment is useful in the treatment of a tumor and/or metastasis.

### CROSS-REFERENCE TO RELATED APPLICATIONS

This application is a Continuation application of U.S. patent application Ser. No. 17/283,482, filed on Apr. 7, 2021, now U.S. Pat. No. ______, which is a U.S. National Stage Application pursuant to 35 U.S.C. § 371 of International Patent Application PCT/EP2019/077116, filed on Oct. 7, 2019, and published as WO 2020/074459 on Apr. 16, 2020, which claims priority to Italy Patent Application 102018000009282, filed on Oct. 9, 2018, all of which are incorporated herein by reference in their entireties for all purposes.

### STATEMENT REGARDING SEQUENCE LISTING

This application contains a sequence listing, which is submitted electronically as an XML formatted sequence listing with a file name “Sequence Listing 689513.0191”, creation date of Dec. 5, 2024, and having a size of 62,587 bytes. sequence listing submitted The electronically is part of the specification and is herein incorporated by reference in its entirety.

### FIELD OF THE INVENTION

The present disclosure concerns a novel antibody-derived therapeutic agent for the treatment of a tumor and/or metastasis.

### BACKGROUND

Cancer is a genetic disease in which somatic endogenous genes undergo mutations. Only a handful of genes—known as oncogenes and tumor suppression genes—are altered in cancer cells and drive tumorigenesis. The activated oncogenes are accelerators and the inactivated tumor suppressor genes are missing brakes for cancer cell growth. Subsequent to the discovery of cancer-causing mutated genes, a new concept emerged for oncogenes: the “oncogene addiction”, which indicates that a cancer cell, despite its abundance of genetic alterations, is still dependent on a single oncogenic protein for its sustained proliferation or survival. Therefore, new therapeutic inroads were undertaken to develop “target therapy” to cure cancer. In the last 20 years, pharmacological targeting of proteins encoded by cancer-causing mutated genes-through chemical drugs or antibodies-has represented the forefront armamentarium to eliminate the mutated cells and combat cancer disease. Target therapies promise to be more effective than conventional cytotoxic chemotherapies, often with fewer side effects. However, only the patients bearing in their tumors the altered specific target gene are likely to benefit from target therapy. Thus, personalized medicine is required in parallel to assess the druggable genetic lesions through comprehensive genomic profiling of the patient tumor.

The MET oncogene encodes for a unique receptor tyrosine kinase endowed with pleiotropic functions. When genetically altered (by point mutations, gene fusion, translocation, and/or amplification), MET initiates transformation of the cells by virtue of its ability to activate the invasive growth program. Thus, MET genetic lesions leading to constitutive Met kinase hyperactivation initiate and maintain the transformed phenotype (“MET addiction”)¹.

MET genetic lesions occur in most solid tumors with an overall frequency of 1-4% and are able to upregulate its kinase activity¹. Point mutations are concentrated in domains critical for Hepatocyte Growth Factor (HGF) ligand binding or receptor signalling (SEMA domain, juxtamembrane domain, and catalytic domain). Most recently, next-generation sequencing revealed exon 14 splice site mutations in 3% of non-small cell lung cancers2, which lead to exon skipping and deletion of the juxtamembrane region of the MET transcript, where a serine residue (Ser985) negatively regulates the Met kinase activity³ and a tyrosine residue (Tyr1003) is required for Met internalization and degradation⁴.

In the “invasive growth” program elicited by MET, the proliferative response is coupled with migration, survival, extracellular matrix degradation, and induction of cell polarity⁵. These biological responses are strived by cells to adapt to adverse conditions and/or escape to find a more convenient environment. In a hostile context, Met is overexpressed—via transcriptional upregulation—by a variety of stimuli such as hypoxia, inflammatory cytokines, pro-angiogenic factors, mitogens and even HGF itself. Lastly, Met is overexpressed in conditions of radiation-induced DNA damage and contributes to resistance to radiotherapy by promoting activation of DNA repair and evasion of programmed cell death of cancer cells.

Several Met-targeting molecules have been developed to erase the hyperactive Met signalling in a selective, robust, and highly effective manner. These drugs include: HGF antagonists (either blocking antibodies or decoys), mAbs targeting the Met receptor, and chemical tyrosine kinase inhibitors (TKIs). Anti-Met mAbs potentially represent a major step in the battle against the cancers driven by MET. Nowadays, four anti-Met mAbs have entered early clinical trials: MetMab (Onartuzumab, Roche), LY2875358 (Emibetuzumab, Eli Lilly & Company), ARGX-111 (Argenx), SAIT301 (Samsung) and Sym015 (Symphogen A/S), a mixture of two antibodies. They act by blocking HGF binding to MET in a competitive fashion (Onartuzumab, ARGX-111) and/or downregulating MET (Emibetuzumab, SAIT301, Sym015).

The murine DN30 mAb (disclosed in WO 2007/090807) is an IgG2A which binds the extracellular domain of the human Met receptor and induces only some of the Met-triggered biological effects⁶. It partially activates receptor phosphorylation due to its bivalent nature which allows simultaneous binding to two distinct antigen molecules, resulting in stabilization of receptor complexes in a fashion similar to that achieved by natural ligands. This unwanted partial agonistic activity on Met was not observed in the monovalent DN30 Fab fragment (MvDN30)⁷. Conversion of the bivalent DN30 parental antibody into the monovalent Fab fragment unleashes the therapeutic potential of the DN30 anti-Met antibody, leading to full antagonist molecule. However, the short half-life of the Fab, due to its low molecular weight, is a severe limitation for the deployment in therapy. The present inventors thus developed new engineered molecules called DCD (Dual Constant Domain Fab) characterized by the duplication of the constant domains present in the DN30 Fab: DCD-1, in which the duplication was done in tandem, and DCD-2, in which the constant domains of the light and heavy chain were reciprocally swapped (disclosed in WO 2014/108829). Both the new recombinant molecules show biochemical properties in vitro comparable to the original Fab, acting as full Met antagonists. In vivo, upon systemic administration, the new recombinant molecules reduce Met-addicted tumor growth. DCD-1 and DCD-2 show a pharmacokinetic profile improved over the original DN30 Fab, nevertheless none of two reach the behavior comparable to the mAb of origin⁸.

It has been previously reported that conversion from bivalent to a monovalent form of an antibody can be reached also by deleting—trough a molecular engineering approach—one of the two antibody arms (disclosed in WO 2005/063816). Data reported an improvement in the in vivo stability of the molecule, mainly due to the activity of the Fc domain, which binds the Fc receptor expressed in the organs.

### SUMMARY OF THE INVENTION

The object of this disclosure is to provide a novel anti-tumor agent derived from a monoclonal antibody useful in the treatment of oncologic patients, namely the DN30 monoclonal antibody.

According to the invention, the above object is achieved thanks to the subject matter recalled specifically in the ensuing claims, which are understood as forming an integral part of this disclosure.

The present invention concerns an anti-Met antibody fragment comprising a single antigen binding arm and an Fc region, wherein the Fc region comprises a complex of a first and a second Fc polypeptide. The antibody fragment comprises:

(i) a first polypeptide comprising one humanized light chain variable (VL) domain, and one human light chain constant (CL) domain, wherein the humanized light chain variable (VL) domain is fused to the human light chain constant (CL) domain in the N-to C-term direction, and wherein the humanized light chain variable (VL) domain contains three complementarity determining regions (CDRs) having the amino acid sequences set forth in SEQ ID No.: 1, 2 and 3, and wherein the humanized VL domain has an amino acid sequence as set forth in SEQ ID No.: 7;

(ii) a second polypeptide comprising one humanized heavy chain variable (VH) domain, one human heavy chain constant CH1 domain and the first Fc polypeptide, wherein the first Fc polypeptide comprises one human hinge region, one human constant CH2 domain and one human constant CH3 domain, wherein the humanized heavy chain variable (VH) domain is fused to the human heavy chain constant CH1 domain, that is fused to the human hinge region, that is fused to the human constant CH2 domain, that is fused to the human constant CH3 domain in the N- to C-term direction, and wherein the humanized heavy chain variable (VH) domain contains three complementarity determining regions (CDRs) having the amino acid sequences set forth in SEQ ID No.: 4, 5 and 6, and wherein the humanized VH domain has an amino acid sequence as set forth in SEQ ID No.: 8;

(iii) a third polypeptide comprising the second human Fc polypeptide, wherein the second human Fc polypeptide comprises one hinge region, one human constant CH2 domain and one human constant CH3 domain, wherein the hinge region is fused to the human CH2 constant domain that is fused to the human CH3 constant domain in the N-to C-term direction, wherein the hinge region is truncated at the N-terminus.

The improved therapeutic properties of the above described antibody fragment f are dependent from the structure of the variable regions, and are not simply due to the monovalent form of the antibody and/or the presence of the Fc region.

The present invention also concerns a product comprising, in a single bottle or in two bottles, (a) the anti-Met antibody fragment as defined above, and a pharmaceutically acceptable vehicle, and (b) an extracellular portion of human Met and a pharmaceutically acceptable vehicle, wherein the extracellular portion of human Met is capable of binding to Hepatocyte Growth Factor (HGF) in a stable manner and contains at least one amino acid mutation within the epitope recognized by the anti-Met antibody fragment to prevent binding of the anti-Met antibody fragment thereto.

### BRIEF DESCRIPTION OF THE DRAWINGS

The invention will now be described in detail, purely by way of an illustrative and non-limiting example and, with reference to the accompanying drawings, wherein:

FIG. 1: Schematic representation of the DN30 antibody and monovalent derivatives. (A) the its original murine bivalent mAb; (B) MvDN30, the chimeric Fab; (C) chOA-DN30, the chimeric monovalent ‘One-Arm’ antibody; (D) HOA-DN30, the humanized monovalent ‘One-Arm’ antibody; (E) The OA-DN30 antibodies were subjected to SDS-PAGE under reducing and not reducing conditions. The gel was stained with Gel Code blue.

FIG. 2: Binding to Met of the monovalent DN30-derived molecules. (A) ELISA binding analysis of MvDN30, chOA-DN30, hOA-DN30 antibodies (liquid phase) to a human Met-Fc chimera (solid phase). (B) ELISA binding analysis of hOA-DN30-cl03E08 (liquid phase) to Met-Fc chimera (solid phase); Met of human, murine, rat, monkey origin were included in the assay. Binding was revealed using anti-human k chain antibodies. O.D.: Optical Density; AU: arbitrary units. Each point is the mean of triplicate values; bars represent standard deviations. Tables below the graphs report Kd values, binding maximum values (Bmax), and the fitting score (R²). (C) Flow-cytometer analysis of hOA-DN30-cl03E08 binding to Met expressed at the surface of a panel of cell lines derived from different species (EBC-1, human lung cancer; C2C12 mouse muscle myoblasts; H9C2 rat cardiac myoblasts; MDCK dog kidney cells; Cos-7 monkey kidney cells). The new molecules bind to human Met-Fc with high affinity; the antibody cross-reacts with human, rat, dog, and monkey Met.

FIG. 3: Agonistic activity of the monovalent DN30-derived molecules. Scatter assay. HPAF-II cells for hrs with the indicated were incubated 24 concentration of HGF (Met ligand), DN30 mAb (partial agonist), MvDN30 (full antagonist) and the chimeric or humanized One-Arm antibodies. All the DN30-derived monovalent molecules do not induce cell scattering, a Met-mediated biological response.

FIG. 4: Met shedding and down-regulation in cells treated by the monovalent DN30-derived molecules. A549 cells were incubated for 48 hrs in serum free medium with the indicated increasing concentration of MvDN30, chOA-DN30, or hOA-DN30 derivatives. Total Met levels were determined by Western blot analysis of cell extracts using anti-Met antibodies. The detected bands correspond to the mature form of the receptor (p145 Met). As a loading control, the filter was probed with an unrelated protein (vinculin). Met shedding was determined by Western blot analysis of conditioned medium using anti-Met antibodies. The detected bands correspond to the shed extracellular domain of the receptor (p80 Met ectodomain). The reports table pixel intensity quantification of each western blot band normalized on the corresponding vinculin signal, as measured by densitometry with ImageJ software. All the monovalent One-Arm antibodies downregulate Met and induce Met shedding.

FIG. 5: Inhibition of Met-activation by the monovalent DN30-derived molecules. GTL-16 (MET-addicted cells carrying a constitutively active Met due to receptor over-expression sustained by MET gene amplification) were incubated for 24 hrs in serum free medium plus hOA-DN30 or chOA-DN30 antibodies (1000 or 250 nM). Met activation was determined in total cell lysates by Western blotting with anti-Met antibodies specific for the phosphorylated Tyr^{1234/1235} Met residues, the major phosphorylation site. The same blot was re-probed with anti-Met antibodies. To control protein loading, the filter was also probed with anti-vinculin antibodies. Chimeric and humanized One-Arm antibodies inhibit Met phosphorylation.

FIG. 6: Vitality of MET-addicted cells treated with the monovalent DN30-derived molecules. GTL-16 human gastric carcinoma cells were plated in 96 well plates in 10% FCS medium. After 24 hrs, cells were treated with increasing concentrations of MvDN30, chOA-DN30, or hOA-DN30 antibodies for a further 72 hrs. Number of cells was evaluated by Cell titer-glo. The plot represents the percentage of alive cells with respect to untreated control. Each point is the mean of triplicate values. The table reports the calculated IC₅₀ values and the fitting score (R²). One-Arm molecules inhibit cell growth of MET-addicted cells.

FIG. 7: Proliferation and death of MET-addicted cells induced by treatment with the monovalent DN30-derived molecules. (A) Proliferation assay: GTL-16 human gastric carcinoma cells were plated in 6 well plates in 10% FCS medium. After 24 hrs, cells were treated with a fixed concentration (1 μM) of MvDN30, chOA-DN30, or hOA-DN30-cl03E08 for a further 48 hrs. EdU (10n μM) was then added to the culture medium for a further 2 hrs. Percentage of cells in S phase was determined by cytofluorimeter analysis following the procedure of the Click-iT EdU Flow Cytometry assay. The plots represent the cells negative and positive for the APC staining (EdU+, S phase cells). Table reports percentage of cells in S phase. (B) Cytotoxicity Assay: GTL-16 human gastric carcinoma cells were plated in 96 well plates in 10% FCS medium. After 24 hrs, cells were treated with increasing concentrations of MvDN30, chOA-DN30, or hOA-DN30-cl03E08 for a further 48 hrs. Cell cytotoxicity was evaluated by Cell-Tox green assay. The graphs represent the increment of cytotoxicity respect to untreated control. Each point is the mean of triplicate values. The table reports the calculated EC₅₀ values, and the fitting score (R²). One-Arm molecule efficiently block proliferation and induce cytotoxicity in MET-addicted cells.

FIG. 8: HGF-induced cell motility in cells treated with hOA-DN30-cl03E08. HPAF-II cells were pre-treated for 18 hrs with the indicated concentrations of hOA-DN30-cl03E08 alone or in combination with DecoyMet^{K842E} (same amount as hOA-DN30-cl03E08) and then incubated for 24 hrs with HGF (6.25 ng/ml). hOA-DN30-cl03E08 inhibits HGF-induced cell motility; the combination with DecoyMet^{K842E} more efficiently counteracts the cell response.

FIG. 9: Invasion of cells treated with hOA-DN30-cl03E08. HPAF-II cells were plated in the upper chamber of transwell filters coated with matrigel™ in serum free medium with 0.5 μM of hOA-DN30-cl03E08 alone or in combination with 1 μM of DecoyMet^{K842E}. Medium with HGF (25 ng/ml) was added in the lower chamber. After 24 hrs, cells that migrated on the lower part of the filter were evaluated by staining and microscope observation. hOA-DN30-cl03E08 inhibits HGF-induced cell invasion; the combination with DecoyMet^{K842E} blocks the biological response.

FIG. 10: Modulation of PD-L1 expression by hOA-DN30-cl03E08. GTL-16 cells were treated with 50 ng/ml of IFN-gamma alone or in combination with 250 nM of hOA-DN30-cl03E08. After 48 hrs, cells were lysed; PD-L1 expression and Met activation on total protein extracts were evaluated by Western blot. As a loading control, filters were probed with anti-GAPDH antibodies.

FIG. 11: Pharmacokinetic profile in vivo of MvDN30, DN30 mAb, and hOA-DN30-cl03E08. Immuno-deficient mice were injected intravenously with a single dose (100 μg) of MvDN30, DN30 mAb, or hOA-DN30. Peripheral blood was collected at different time points. Serum concentrations of the therapeutic molecules were measured by ELISA. Graph represents the amount of circulating molecules as a function of time. Samples are in triplicate; bars represent standard deviations.

FIG. 12: Inhibition of MET-addicted tumor growth in vivo by treatment with the monovalent DN30 derivatives. 1×10⁶ GTL-16 MET-addicted gastric carcinoma cells were inoculated in the flank of immune-deficient NOD-SCID mice. When the tumors reached an average volume of 68.4±5.4 mm³, mice were randomized in 4 homogeneous groups and treated with 30 mg/kg of MvDN30, chOA-DN30, or hOA-DN30-cl03E08 by intravenous injection 2 times per week. As a control, a group was treated with the same volume of PBS (Vehicle). Tumor growth was monitored periodically with a caliper. After 15 days, mice were sacrificed. (A) Kinetics of tumor growth; (B) Tumor volumes at the end of the experiment reported as percentage of the untreated group. hOA-DN30-cl03E08 inhibits MET-addicted tumor growth more efficiently than the chOA-DN30.

FIG. 13: Dose-response inhibition of MET-addicted tumor growth in vivo by hOA-DN30-cl03E08 treatment. 1×10⁶ GTL-16 MET-addicted gastric carcinoma cells were inoculated in the flank of immunodeficient NOD-SCID mice. After 8 days, the tumors reached an average volume of 80.7±2 mm³ and the mice were randomized in 5 homogeneous groups and treated with increasing concentrations of hOA-DN30-cl03E08 by intravenous injection. As a control, a group was treated with the same volume of PBS (Vehicle). Tumor growth was monitored periodically with a caliper. After 17 days, mice were sacrificed, and the tumors were excised and weighted. (A) kinetics of tumor growth; (B) Tumor weights. hOA-DN30-cl03E08 inhibits MET-addicted tumor growth with a dose-response profile.

FIG. 14: Amino acid sequences of VL, VH, CL and CH1 domains of chOA-DN30 and hOA-DN30-cl03E08 antibodies. The Complementarity Determining Regions sequences are highlighted by a bold, underlined character.

FIG. 15: Amino acid sequences of the first and the second Fc polypeptides of OA-DN30. The amino acid mutations within the CH3 domains of the first and second Fc polypeptides—necessary for generating the knob and the hole, respectively,—are highlighted by a bold, underlined character.

FIG. 16: Amino acid sequence of the mutated form of DecoyMet. The amino acid mutation K842E is highlighted by a bold, underlined character.

FIG. 17: A copy of the Viability Statement for the biological material “DN30” due to the deposit on Apr. 16, 2008, of material assigned Deposit Number PD08003.

FIG. 18: A copy of the Receipt in the Case of an Original Deposit of the biological material “DN30” due to the deposit on Dec. 29, 2005, of material assigned Deposit Number PD 05006.

### DETAILED DESCRIPTION OF THE INVENTION

In the following description, numerous specific details are given to provide a thorough understanding of the embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret the scope or meaning of the embodiments.

The instant disclosure concerns a novel therapeutic agent for the treatment of a tumor and/or metastasis.

It is estimated that more than 200,000 patients per year are “MET-addicted”, and Met inhibition may potentially result in remission of the disease.

Considering that mutations accumulate with aging and over the next 20 years the ageing population will increase, the burden of cancer is expected to rise and to have a high impact on global healthcare resources for patients' management. Genetic alterations responsible for “MET-addiction” have been found in gastric, oesophageal, colorectal, renal, and lung carcinoma, melanoma, and brain tumors (see COSMIC database: sanger.ac.uk). Moreover, selection of MET genetic lesions has been found as an acquired mechanism of resistance to a number of other targeted therapies in colorectal and non-small cell lung cancer (NSCLC)⁹.

The role of Met in metastasis is also associated with the ability of Met in helping cells to adapt to a harsh environment. The metastatic capabilities driven by Met not only depend on genetic and epigenetic alterations, but also on paracrine secretion of HGF by tumor stromal tissue which is composed by a large variety of cell types, including fibroblasts, resident epithelial cells, pericytes, myofibroblasts, vascular and lympho-vascular endothelial cells, and infiltrating cells of the immune system.

Met is nowadays recognized as a cancer-specific target for: (i) personalized treatment of tumors with MET mutations/amplifications (‘addicted to MET’); (ii) for prevention/reversion of Met-driven primary and secondary resistance to other targeted cancer therapies; and (iii) for prevention/reversion of Met-driven invasive/metastatic phenotype¹.

The DN30 inhibitory activity lies on its ability to promote the physiological activity of the metalloprotease ADAM-10 acting at the cell surface¹⁰, which releases from the plasma membrane the intact extracellular portion of the receptor (Met-shedding), followed by the rapid degradation of the Met intracellular kinase domain¹¹. As a consequence of these processes, Met is physically removed from the cell surface and the extracellular domain acts as soluble ‘decoy’, preventing ligand binding by sequestering HGF. Thus, the advantage of DN30 is to inhibit both HGF-dependent and HGF-independent activation of Met¹².

As compared with other anti-Met antibodies, DN30 blocks the receptor functions by a unique mechanism that rise to different and synergic activities: (i) removal of Met from the cell surface by ‘shedding’ of the ectodomain; (ii) sequestration of the HGF ligand; (iii) inhibition of the homo-or hetero-dimerization of Met receptor at the membrane; (iv) stimulation of receptor degradation. Thus, DN30 offers the opportunity to kill two birds with one stone, allowing optimal blockade of the Met pathway, as it can potentially act not only on cancer cells but also on the tumor microenvironment (HGF-driven biological responses of endothelial cells, fibroblasts, and macrophages).

The present invention relies on the idea that, to reach the highest therapeutic potential against a tumor by means of an anti-Met antibody, it is mandatory to apply a molecule that: i) is not eliciting, even also responses evoked upon Met in part, intracellular activation, i.e. is not an agonist or a partial agonist; ii) impairs Met activation through a mechanism of action different from ligand (HGF) displacement; iii) has a favorable pharmacokinetic profile, suitable for deployment in the clinic; iv) reasonably does not exert immune-response in humans. To fulfill all the above considerations, the inventors converted the murine DN30 antibody-that blocks Met activation by a shedding mechanism-into a fully humanized, monovalent and highly stable molecule, by formatting it in the “One-Arm” form, named in the following as “hOA-DN30”.

In an embodiment, the present invention concerns an anti-Met antibody fragment comprising a single antigen binding arm and an Fc region, wherein the Fc region comprises a complex of a first and a second Fc polypeptide. The antibody fragment comprises, preferably consists of:

(i) a first polypeptide comprising, preferably consisting of, one humanized light chain variable (VL) domain, and one human light chain constant (CL) domain, wherein the humanized light chain variable (VL) domain is fused to the human light chain constant (CL) domain in the N-to C-term direction, and wherein the humanized light chain variable (VL) domain contains three complementarity determining regions (CDRs) having the amino acid sequences set forth in SEQ ID No.: 1, 2 and 3, and wherein the humanized light chain variable (VL) domain has an amino acid sequence as set forth in SEQ ID No.: 7;

(ii) a second polypeptide comprising one humanized heavy chain variable (VH) domain, one human heavy chain constant CH1 domain and the first Fc polypeptide, wherein the first Fc polypeptide comprises one human hinge region, one human constant CH2 domain and one human constant CH3 domain, wherein the humanized heavy chain variable (VH) domain is fused to the human heavy chain constant CH1 domain, that is fused to the human hinge region, that is fused to the human constant CH2 domain, that is fused to the human constant CH3 domain in the N- to C-term direction, and wherein the humanized heavy chain variable (VH) domain contains three complementarity determining regions (CDRs) having the amino acid sequences set forth in SEQ ID No.: 4, 5 and 6, and wherein the humanized heavy chain variable (VH) domain has an amino acid sequence as set forth in SEQ ID No.: 8.;

(iii) a third polypeptide comprising, preferably consisting of, the second human Fc polypeptide, wherein the second human Fc polypeptide comprises, preferably consists of, one human hinge region, one human constant CH2 domain and one human constant CH3 domain, wherein the hinge region is fused to the human CH2 constant domain, that is fused to the human CH3 constant domain in the N-to C-term direction, wherein the hinge region is truncated at the N-terminus.

In one embodiment, the human light chain constant (CL) domain is a human light kappa type domain.

In one embodiment, the human hinge region and the human constant domains CH1, CH2 and CH3 are from a human IgG1.

In one embodiment, the two Fc polypeptides are linked through intermolecular disulfide bonds at the hinge region.

In one embodiment, the first Fc polypeptide and the second Fc polypeptide meet at an interface, and one between the first and the second Fc polypeptide comprises a knob at the interface, and the other between the first and the second Fc polypeptide comprises a hole at the interface, wherein the knob is positionable into the hole.

In a preferred embodiment, one between the first and the second Fc polypeptide comprises a mutated CH3 constant domain, wherein the mutated CH3 constant domain carries an amino acid mutation at position 389, wherein the original amino acid at position 389 has been mutated to import an amino acid having a larger side chain volume than the original amino acid, so as to create a knob at the interface between the Fc polypeptides; and wherein the other between the first and the second Fc polypeptide comprises a mutated CH3 constant domain, wherein the mutated CH3 constant domain carries three amino acid mutations at positions 389, 391 and 438, wherein the original amino acids have been mutated to import amino acids having smaller side chain volumes than the original amino acids, so as to create a hole at the interface between the Fc polypeptides, wherein the knob is positionable into the hole, and wherein the amino acid numbering is according to the EU numbering scheme of Kabat (pp. 688-696 in Sequences of proteins of immunological interest, 5th ed., Vol. 1, 1991; NIH, Bethesda, MD).

In a still preferred embodiment, the original amino acids at positions 389, 391 and 438 are threonine, leucine and tyrosine respectively; and wherein in one between the first and the second Fc polypeptide the threonine in position 389 has been mutated to tryptophan; and wherein in the other between the first and the second

Fc polypeptide the threonine at position 389 has been mutated to serine, the leucine at position 391 has been mutated to alanine and the tyrosine at position 438 has been mutated to valine.

In a preferred embodiment, the human light chain constant (CL) domain has an amino acid sequence as set forth in SEQ ID No.: 9 and the human heavy chain constant CH1 domain has an amino acid sequence as set forth in SEQ ID No.: 10.

In a preferred embodiment, the first human Fc polypeptide has an amino acid sequence as set forth in SEQ ID No.: 11, and the second human Fc polypeptide has an amino acid sequence as set forth in SEQ ID No.: 12.

In an embodiment, the present invention concerns an isolated nucleic acid encoding the anti-Met antibody fragment as disclosed above.

In an embodiment, the present invention concerns a composition comprising two or more recombinant nucleic acids which collectively encode the anti-Met antibody fragment as disclosed above.

The anti-Met antibody fragment disclosed herein when bound to Met induces shedding of the extracellular domain of Met. The anti-Met antibody fragment when bound to Met does not exert an agonistic activity towards Met. The anti-Met antibody fragment when bound to Met inhibits Met phosphorylation. The anti-Met antibody fragment when bound to Met further blocks proliferation and induces cytotoxicity in MET-addicted cells. The anti-Met antibody fragment when bound to Met further inhibits cell motility and cell invasion induced by Hepatocyte Growth Factor (HGF). The aforementioned properties of the anti-Met antibody fragment object of the instant description provide said antibody fragment with anti-tumor and/or anti-metastasis activity.

The expression “antigen binding arm”, as used herein, refers to a component part of an antibody fragment of the invention that has an ability to bind specifically a target molecule of interest. The antigen binding arm is a complex of variable domain sequences (VL and VH), including the CDRs and the Frame Regions of an immunoglobulin light and heavy chain, and constant domain sequences (CL and CH) of an immunoglobulin light and heavy chain.

“Humanized” forms of non-human (e.g., murine) antibodies are non-natural recombinant antibodies that contain minimal amino acid residues derived from non-human immunoglobulin. For the most part, humanized antibodies are human immunoglobulins (recipient antibody) in which residues from a hypervariable region of the recipient (i.e. the Complementarity Determining Regions-CDRs) are replaced by residues from a hypervariable region of a non-human species (donor antibody) having the desired specificity, affinity, and properties. In some instances, framework region (FR) residues of the human immunoglobulin can be replaced by corresponding non-human residues.

Furthermore, humanized antibodies may comprise residues that are not found in the recipient antibody or in the donor antibody. These modifications are made to further refine antibody performance. In general, the humanized antibody will comprise two variable domains, in which all or substantially all of the hypervariable loops correspond to those of a non-human immunoglobulin and all or substantially all of the Frame residues (FRs) are those of a human immunoglobulin sequence. The humanized antibody also comprises a portion of an immunoglobulin constant region (Fc), typically that of a human immunoglobulin. For further details, see ¹³⁻¹⁵. See also the following review articles and references cited therein: ¹⁶⁻¹⁸.

The term “Fc region”, as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain might vary, the human IgG1 heavy chain Fc sequence is usually defined to stretch from an amino acid residue included between Asp 234 and Thr 238, to the carboxyl terminus of the Fc sequence according to the EU numbering scheme of Kabat. The Fc sequence of an immunoglobulin generally comprises one hinge region, two constant domains, a CH2 domain and a CH3 domain, and optionally comprises a CH4 domain. By “Fc polypeptide” herein is meant one of the polypeptides that make up an Fc region. The Fc region increases stability of the antibody fragment compared to a Fab molecule comprising an antigen binding arm.

The “hinge region”, “hinge sequence”, and variations thereof, as used herein, includes the meaning known in the art, which is illustrated in, for example, Janeway et al., Immuno Biology: the immune system in health and disease, (Elsevier Science Ltd., NY) (4th ed., 1999) and in ^{19,20}.

The phrase “truncated hinge region”, as used herein, refers to a polypeptide comprising parts, but not all, of a hinge sequence. The truncated hinge region is capable of linkage to the “first” Fc polypeptide. If the wild type hinge sequence is not present, the remaining sequence in the “second” Fc polypeptide would comprise a component that is capable of linkage to the “first” Fc polypeptide. For example, said component can be a modified residue or an added cysteine residue capable of forming a disulfide linkage.

A “knob” refers to at least one amino acid side chain which projects from the interface of a first Fc polypeptide and is therefore positionable in a compensatory hole in the adjacent interface (i.e. the interface of a second Fc polypeptide) so as to stabilize the heteromultimer, and thereby favor heteromultimer formation over homomultimer formation, for example. The knob may exist in the original interface or may be introduced synthetically (e.g. by altering nucleic acid the interface). Normally, a nucleic acid encoding encoding the interface of the first polypeptide is altered to encode the knob. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The upper limit for the number of original residues which are replaced is the total number of residues in the interface of the first polypeptide.

A “hole” refers to at least one amino acid side chain which is recessed from the interface of a second Fc polypeptide and therefore accommodates a corresponding knob on the adjacent interface of a first Fc polypeptide. The hole may exist in the original interface or may be introduced synthetically (e.g. by altering nucleic acid encoding the interface). Normally, nucleic acid encoding the interface of the second polypeptide is altered to encode the hole. To achieve this, the nucleic acid encoding at least one “original” amino acid residue in the interface of the second polypeptide is replaced with nucleic acid encoding at least one “import” amino acid residue which has a smaller side chain volume than the original amino acid residue. It will be appreciated that there can be more than one original and corresponding import residue. The upper limit for the number of original residues which are replaced is the total number of residues in the interface of the second polypeptide.

The knob is “positionable” into the hole which means that the spatial location of the knob and hole on the interface of a first Fc polypeptide and second Fc polypeptide respectively and the sizes of the knob and hole are such that the knob can be located into the hole without significantly perturbing the normal association of the first and second polypeptides at the interface. Since knobs do not typically extend perpendicularly from the axis of the interface and have preferred conformations, the alignment of a knob with a corresponding hole relies on modeling the knob/hole pair based upon a three-dimensional structure such as that obtained by X-ray crystallography or nuclear magnetic resonance (NMR). This can be achieved using widely accepted techniques in the art.

The expression “non-agonist activity” of the anti-Met antibody fragment as used herein refers to the anti-Met antibody fragment that does not exert any activity that elicits, even also in part, cellular responses evoked upon Met activation. The non-agonistic activity of the anti-Met antibody fragment can be measured by the evaluation of the Met level of phosphorylation by conventional techniques such as western blot, immunofluorescence, immunohistochemistry, ELISA, cytofluorimeter analysis or any other method that includes the use of an antibody that recognizes specifically Met, or the Met residues Tyr¹²³⁴⁻¹²³⁵ if phosphorylated (i.e. the major phosphorylation site of Met²¹), or the Met residues Tyr^{1349/1356} if phosphorylated (i.e. the docking site of Met²²) upon incubation of the cells with the said non-agonist antibody. As an alternative, the evaluation of cellular biological responses upon antibody treatment compared to the responses induced by HGF treatment can be applied.

The present invention further concerns a product comprising, in a single bottle or in two bottles, (a) the anti-Met antibody fragment as disclosed herein, and a pharmaceutically acceptable vehicle, and (b) an extracellular portion of human Met and a pharmaceutically acceptable vehicle, wherein the extracellular portion of human Met is capable of binding to Hepatocyte Growth Factor (HGF) in a stable manner and contains at least one amino acid mutation within the epitope recognized by the anti-Met antibody fragment to prevent binding of the anti-Met antibody fragment thereto.

In an embodiment, the extracellular portion of human Met contains SEMA, PSI, IPT-1, IPT-2, IPT-3 and IPT-4 domains.

In a preferred embodiment, the extracellular portion of human Met has an amino acid sequence as set forth in SEQ ID No.: 13, wherein at least one of the amino acids between position 797 and position 875 of SEQ ID No.: 13 is mutated in order to prevent binding of the anti-Met antibody fragment thereto.

In a more preferred embodiment, the extracellular portion of human Met has the amino acid sequence set forth in SEQ ID No.: 14.

The present description also concerns a nucleic acid molecule encoding the extracellular portion of human Met capable of binding to Hepatocyte Growth Factor (HGF) in a stable manner and containing at least one amino acid mutation within the epitope recognized by the anti-Met antibody fragment to prevent binding of the anti-Met antibody fragment thereto, preferably the extracellular portion of human Met having the amino acid sequence set forth in SEQ ID No.: 14.

As used herein, the expression “the extracellular portion of human Met is capable of binding to human HGF in a stable manner” means that the extracellular portion of human Met binds to HGF with a calculated Kd not higher than 100 nM.

The expression “the extracellular portion of human Met contains at least one amino acid mutation at the epitope recognized by the anti-Met antibody fragment”, as used herein, means the presence of one or more amino acid mutations (i.e. substitutions and/or deletions and/or insertions) within the extra-cellular portion of Met, able to induce a modification within the extra-cellular portion of Met that prevents the engagement of the above region by the anti-Met antibody variable domains. The skilled man in view of his common general knowledge (represented i.a. by the possibility to generate a cDNA including a single nucleotide change in a given DNA sequence using specific primers during DNA duplication; see Maniatis T. Molecular cloning: A laboratory manual Cold Spring Harbor Laboratory, 1982) does not need further details about the realization of a mutated form of the extracellular portion of human Met retaining the ability to bind to human HGF but not to the anti-Met antibody fragment. The present invention must not therefore be interpreted as encompassing only the mutated extra-cellular portion of human Met as disclosed herein (i.e. SEQ ID No.: 14), since the skilled man in view of the common general knowledge can produce further mutated versions of the extra-cellular portion of human Met having SEQ ID No.: 13 that prevent the bind of the anti-Met antibody thereto.

The terms “SEMA”, “PSI”, “IPT-1”, “IPT-2”, “IPT-3” and “IPT-4” refer to the Met domains constituting the extracellular region of Met. Such domain names belong to the common general knowledge of a skilled man as represented i.a. by ^{23,24}. SEMA domain is a protein interacting module in common with semaphorins and plexins encompassing the region comprised between amino acids 25-516 of Met (SEQ ID No.: 13); PSI is a domain in common with Plexins, Semaphorins, and Integrins encompassing the region comprised between amino acids 519-561 of Met (SEQ ID No.: 13); the IPT domain—repeated four times—is a region Immunoglobulin-like in common with Plexins and Transcription factors, encompassing the region comprised between amino acids 563-934 of MET (SEQ ID No.: 13). In detail, IPT repeat 1 covers the amino acid positions 563-656 of SEQ ID No.: 13, IPT repeat 2 covers the amino acid positions 657-740 of SEQ ID No.: 13, IPT repeat 3 covers the amino acid positions 741-837 of SEQ ID No.: 13, IPT repeat 4 covers the amino acid positions 838-934 of SEQ ID No.: 13.

The present invention further concerns the therapeutic use of the anti-Met antibody fragment as disclosed herein, optionally in combination with an extracellular portion of human Met as disclosed herein, for use in the treatment of a patient suffering from a tumor and/or metastasis, wherein the patient carries genetic alterations of the MET gene.

The present invention further concerns the therapeutic use of the anti-Met antibody fragment as disclosed herein in combination with an extracellular portion of human Met as disclosed herein for use in the treatment of a patient suffering from a tumor and/or metastasis, wherein the patient carries a wild-type MET gene.

In one aspect, the invention provides a method of treating a tumor and/or metastasis in a subject, said method comprising administration to the subject of an effective amount of the anti-Met antibody fragment, optionally in combination with an extracellular portion of human Met, whereby said condition is treated.

In one aspect, the invention provides a method of inhibiting the growth of a cell that expresses Met, said method comprising contacting said cell with the anti-Met antibody fragment disclosed herein, optionally in combination with an extracellular portion of human Met of the invention, thereby causing an inhibition of growth of said cell.

In one aspect, the invention provides a method of therapeutically treating a mammal having a cancerous tumor and/or metastasis, said method comprising administration to said mammal of an effective amount of the anti-Met antibody fragment disclosed herein, optionally in combination with an extracellular portion of human Met, thereby effectively treating said mammal.

In one aspect, the invention provides a method for treating a cell proliferative disorder, said method comprising administration to a subject in need of such treatment of an effective amount of the anti-Met antibody fragment object of the present disclosure, optionally in combination with an extracellular portion of human Met, thereby effectively treating or preventing said cell proliferative disorder.

In one aspect, the invention provides a method of therapeutically treating a tumor and/or metastasis in a mammal, wherein the growth of said tumor is at least in part dependent upon a growth potentiating effect of the Met/HGF system, as the consequence of either an increase in cell proliferation, a protection from apoptosis, or both. Said method comprises contacting a tumor cell with an effective amount of the anti-Met antibody fragment, optionally in combination with an effective amount of an extracellular portion of human Met of the invention, thereby effectively treating said tumor and/or metastasis.

The tumor, that can be effectively treated with the anti-Met antibody, optionally in combination with an extracellular portion of human Met, is selected from breast, colorectal, lung, colon, pancreatic, prostate, ovarian, cervical, central nervous system, renal, hepatocellular, bladder, gastric, head and neck tumor cell, papillary carcinoma (e.g. the thyroid gland), melanoma, lymphoma, myeloma, glioma/glioblastoma (e.g. anaplastic astrocytoma, multiforme glioblastoma, anaplastic oligodendroglioma, anaplastic oligodendroastrocytoma), leukemia cell, sarcoma, rhabdomyosarcoma, or a tumor from a Cancer of Unknown Primary origin (CUP).

In one embodiment, a cell that is therapeutically targeted in a method of the invention is a hyperproliferative and/or hyperplastic cell. In one embodiment, a cell that is targeted in a method of the invention is a dysplastic cell. In yet another embodiment, a cell that is targeted in a method of the invention is a metastatic cell. In a further embodiment, a cell that is targeted in a method of the invention is a Met expressing cell belonging to the microenvironment sustaining the tumor and/or the metastasis.

The present inventors demonstrated that the One-Arm structure, obtained by selected point mutations of the CH3 domain and resulting in efficient Fc heterodimerization, originates an antibody into a monovalent form.

Monovalency is essential to avoid the eventual agonistic activity of the DN30 due to the bivalent native structure of the antibody.

Unexpectedly and surprisingly the present inventors discovered that one of the humanized One-Arm format of the DN30 (i.e. hOA-DN30-cl03E08) displays strong inhibitory properties in vitro and in vivo.

In vitro hOA-DN30-cl03E08 shows a superior activity as compared to the corresponding chimeric form (named in the following as “chOA-DN30”) and to all the other humanized One-Arm derivatives herein disclosed (named “hOA-DN30 antibodies”) (see Material and Methods section for a detailed description of all the One-Arm antibodies derived from DN30). Unexpectedly hOA-DN30-cl03E08 binds to Met with a higher affinity as compared to the other OA-DN30 antibodies tested, either chimeric or human. Unpredictably, the inventors found that the shedding response is potentiated by increasing the affinity of the DN30 antibody for Met. Consequently, the inhibition of Met activation, measured as amount of phosphorylated Met receptors, was stronger. These activities translate into a repression of Met-mediated biological responses. Impairment of MET-addicted tumor cell growth occurs at very low dose; the hOA-DN30-cl03E08 IC₅₀ is the lowest among all those measured for the OA-DN30 antibodies included in the analysis, either chimeric or human. A better inhibition of tumor cell growth can be due to a more extensive block of cell proliferation, and/or to a more pronounced increment of cytotoxicity.

The hOA-DN30-cl03E08 shows improved therapeutic potential also in vivo. This is expected if the comparison is done with MvDN30, a molecule that, due to its low molecular weight, is subjected to high kidney clearance. The poor pharmacokinetic properties of MvDN30 considerably affect the performance of the molecule in the in vivo experimental models.

One can consider that reaching a molecular weight higher than the cut-off of renal clearance is per se sufficient to elicit the best therapeutic response, but this is not the case. In fact, the results obtained with the DCD-1 and DCD-2 derivatives of the DN30 mAb—recombinant molecules with increased size with respect to MvDN30—clearly show that the pharmacokinetic profile is only partially improved and the therapeutic response is limited to a reduction of the tumor growth rate⁸.

The superior activity of the hOA-DN30-cl03E08 can in part be due to the presence of the Fc region that generates differences in the tissue/organ distribution of the antibody due to the binding to the Fc Receptor, thus providing a highly favourable pharmacokinetic profile.

The positive aspects related to the in vivo stability due to the presence of the Fc region have been already discussed and disclosed during Onartuzumab application. This is an anti-Met antibody designed in the One-Arm format²⁵. Nevertheless this antibody is produced in bacteria, thus is not glycosylated. Consequently, it lacks the ability to activate Antibody Dependent Cellular Cytotoxicity (ADCC) and/or Complement Dependent cytotoxicity (CDC). On the contrary, hOA-DN30-cl03E08 is produced in mammalian cells and therefore, it must be considered fully able to elicit Fc effector functions.

hOA-DN30-cl03E08 is different from Onartuzumab also for the mechanism of action. While the first antibody induces Met receptor shedding, wiping out Met from the cell surface and sponging HGF in the extracellular environment, the second one competes with HGF for Met binding. As result, hOA-DN30 inhibits Met independently from the mechanisms that sustain the aberrant activation of the onco-receptor, while Onartuzumab is effective only in the cases of ligand-dependent activation.

The presence of the Fc region cannot be responsible of the notable improvement of the therapeutic response in vivo obtained by the hOA-DN30-cl03E08, as this domain is present in all the humanized and chimeric One-Arm antibody tested. What the humanized and the chimeric One-Arm antibodies do not share are the variable regions, in which lies the binding site of the antibodies. In particular, the DN30 CDRs—identical in all the derivatives—are inserted in the frame regions. These parts of the antibody, even if not directly involved in the formation of the binding pocket, include particular amino acids, called Vernier residues²⁶, that could influence the final conformation of the CDR structures and therefore could determine the final affinity of the antibody for the antigen. The DN30 CDRs in combination with the frame sequences, i.e. the sequences of the heavy and light variable domains, of the cl03E08 originate an anti-Met antibody with a stronger affinity probably because the sequence combination generates a binding pocket fitting better with the corresponding epitope on the Met receptor. A more robust interaction between the antibody and Met further implements the shedding of the receptor and the consequent impairment of Met-mediated biological responses. This, conjugated with the improved plasma stability due to the One-Arm format, gives rise to an antibody—i.e. the cl03E08—with unexpected superior activity against MET-addicted/driven cancers.

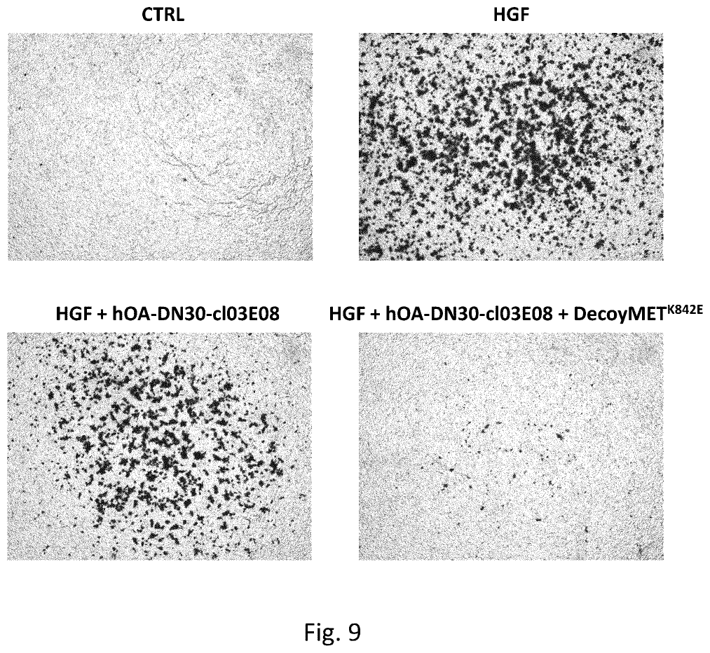

Data reported in Basilico et al. ²⁷ show that concomitant targeting of both HGF and Met gives rise to a higher therapeutic robustness in the treatment of tumors and/or metastasis. The experimental data witnessed herein unexpectedly show that inhibition of the MET/HGF axis by means of hOA-DN30 in combination with DecoyMet^{K842E} is superior to the one obtained upon the application of MVDN 30 in combination with DecoyMet^{K842E}. In fact, comparing the results obtained in the invasion assay (here reported in FIG. 9) with the ones published in Basilico et al. ²⁷, hOA-DN30 abrogates the HGF-driven invasion of cancer cells, while a residual activity is still present if MvDN30 is part of the combination.

Cancer cells without MET genetic alterations exploit the ‘physiological’ program triggered by the MET oncogene as an ‘expedient’ to boost the malignant phenotype and to unleash the invasive metastatic phenotype in response to stress conditions such as hypoxia, ionizing radiation or chemotherapy. ‘Expedience’ requires stimulation of wild-type MET by its ligand HGF. The data provided herein show that, in conditions of MET ‘expedience’, a concomitant intervention hitting both sides of the MET/HGF axis results in improved inhibitory activity. The hOA-DN30-cl03E08 induces the physical removal of MET from the cell surface by ‘shedding’ of the ectodomain. The latter is released in the extracellular environment and acts as ‘decoy’ for HGF. Exogenous supply of recombinant DecoyMet reinforces the HGF-sequestering activity of the endogenous DecoyMet generated by hOA-DN30-cl03E08. The combination of hOA-DN30-cl03E08 and DecoyMet^{K842E} acts simultaneously on Met-expressing cancer cells and on HGF-secreting tumor stroma. This allows optimal blockade of the HGF-driven Met signalling and thus represents a reliable therapeutic option for a large cohort of patients carrying tumors expressing wild-type MET that rely on MET for sustaining the invasive/metastatic phenotype.

The proteins disclosed herein (i.a. the humanized anti-Met antibody and the extracellular portion of human Met) can be easily manufactured either in the form of proteins or in the form of nucleic acid molecules encoding the proteins by a skilled man in view of the common general knowledge of the field related to the recombinant DNA technology, as represented i.a. by Maniatis T. Molecular cloning: A laboratory manual Cold Spring Harbor Laboratory (1982). For example, the following standard procedure can be followed: (i) synthesis of the corresponding cDNA sequences, (ii) insertion of the CDNAs into a plasmid suitable for expression in mammalian by conventional recombinant DNA methods, (iii) transient or stable co-transfection with the above mentioned plasmids of a mammalian cell line, (iv) collection of the culture supernatant, (v) purification by chromatography of the affinity recombinant protein.

Therapeutic compositions comprising the active ingredient(s) of the instant invention, i.e. the humanized anti-Met antibody fragment alone or in combination with an extracellular portion of human Met, can be prepared with physiologically acceptable carriers, excipients, or stabilizers (Remington's Pharmaceutical Sciences 16th edition, Osol, A. Ed., 1980), in the form of aqueous solutions, lyophilized, or other dried formulations. Acceptable carriers, excipients, or stabilizers are nontoxic to recipients at the dosages and concentrations employed, and include buffers; antioxidants; preservatives; low molecular weight (less than about 10 residues) polypeptides; proteins, such as serum albumin, gelatin, or immunoglobulins; hydrophilic polymers such as polyvinylpyrrolidone; amino acids; monosaccharides, disaccharides, and other carbohydrates; chelating agents; sugars; salt-forming counter-ions; metal complexes and/or non-ionic surfactants. The formulations may also contain other active compound(s) as necessary for the particular indication being treated, preferably those with complementary activities that do not adversely affect the therapeutic activity of hOA-DN30 alone or in combination with the extracellular portion of human Met. Such molecules are suitably present in combination in amounts that are effective for the purpose intended.

The active ingredient(s) may also be entrapped in microcapsules prepared by means of techniques disclosed i.a. in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the active ingredients of the invention, which matrices are in the form of shaped articles, e.g. films, or microcapsule.

The active ingredient(s) of the invention can be either alone or in combination with another used antibody, small molecule tyrosine kinase inhibitors, chemotherapeutic agent(s) (including cocktails of chemotherapeutic agents), other cytotoxic agent(s), anti-angiogenic agent(s), cytokines, and/or growth inhibitory agent(s). Such combined therapies noted above include combined administration (where the two or more agents are included in the same or separate formulations), and separate administration, in which case, administration of hOA-DN30 can occur prior to, and/or following, administration of the adjunct therapy or therapies.

The active ingredient(s) of the present invention (and adjunct therapeutic agent(s)) is (are) administered by any suitable means, including parenteral, subcutaneous, intraperitoneal, intrapulmonary, and intranasal, and, if desired for local treatment, intralesional administration. The active ingredient(s) of the instant invention can be suitably administered by pulse infusion, particularly with declining doses of the active ingredients. Dosing can be by any suitable route, e.g. by injections, such as intravenous or subcutaneous injections, depending in part on whether the administration is brief or chronic.

The active ingredient(s) of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The active ingredient(s) of the invention need not be, but may optionally be, formulated with one or more agents currently used to prevent or treat the disorder in question. The effective amount of such other agents depends on the amount of active ingredient(s) of the invention present in the formulation, the type of disorder or treatment, and other factors discussed above. These are generally used in the same dosages and with administration routes as used hereinbefore or about from 1 to 99% of the heretofore employed dosages.

For the treatment of disease, the appropriate dosage of the active ingredient(s) of the invention (when used alone or in combination with other agent(s) such as chemotherapeutic agent(s)) will depend on the type of disease to be treated, the severity and course of the disease, whether the active ingredients are administered for preventive or therapeutic purposes, the patient's clinical history and response to the active ingredients of the invention are duly taken into consideration, and at the discretion of the attending physician.

hOA-DN30 is suitably administered to the patient at one time or over a series of treatments. Depending on the type and severity of the disease, about 1 mg/kg to 30 mg/kg of antibody is an initial candidate dosage for administration to the patient, whether, for example, by one or more separate administrations, or by continuous infusion. One typical daily dose might range from about 1 μg/kg to 100 mg/kg or more, depending on the factors mentioned above. For repeated administrations over several days or longer, depending on the condition, the treatment is sustained until a desired suppression of disease symptoms occurs. One exemplary dosage of the antibody fragment would be in the range from about 0.05 mg/kg to about 20 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays. With respect to the combination therapy, the extracellular portion of human Met is preferably delivered at the same time of hOA-DN30 and dosed in proportion to hOA-DN30, preferentially but not limited to, the molar ratio 1:1 hOA-DN30: DecoyMet^{K842E}, depending on the type and severity of the disease.

### Results

### Generation of the Chimeric and Humanized One-Arm Formats of the DN30 Antibody

Through classical molecular biology techniques, the present inventors substituted the constant domains of the DN30 mAb heavy and light chains with constant domains derived from human immunoglobulins. The light chain constant domain was substituted with the human kappa type domain, (the one more represented in the natural human antibodies), while the heavy chain constant domains were substituted with the homologous domains derived from the human IgG1 (the one that can induce ADCC/CDC).

Humanization of the DN30 mouse antibody has been done by Fair Journey Biologics using a phage display library approach. They started from the identification of deviating FR (frame) residues and the analysis of the closest human V germline, chosen from germlines with identical canonical fold combination for CDR1-CDR2, using available tools and databases: abYsistool from Dr. Andrew Martin at UCL plus, bioc.uzh.ch/plueckthun/antibody/and mrc-lmb.cam.ac.uk/vbase/. Upon generation of phage display libraries—in which randomization of the VH and VL region has been performed—four affinity-driven phage display rounds of selection have been done. At the end of the process, the DN30 humanized variants with the highest human identity and homology were selected. Variable regions were linked through classical molecular biology techniques to the constant domain sequences derived from human immunoglobulins, i.e. human kappa light chain and IgG1 heavy chain constant domains.

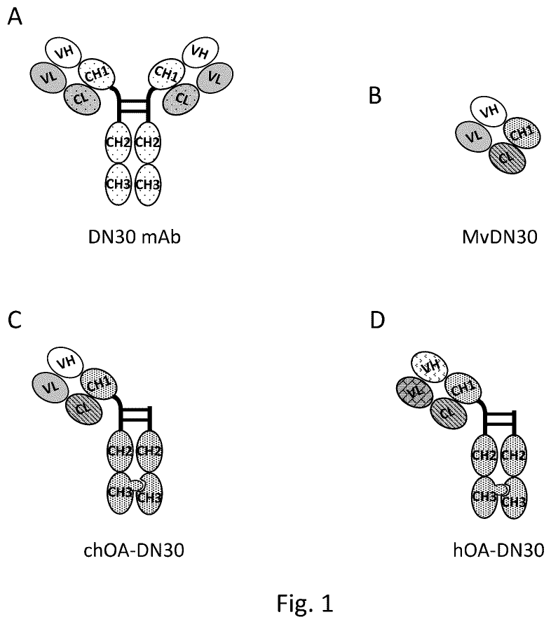

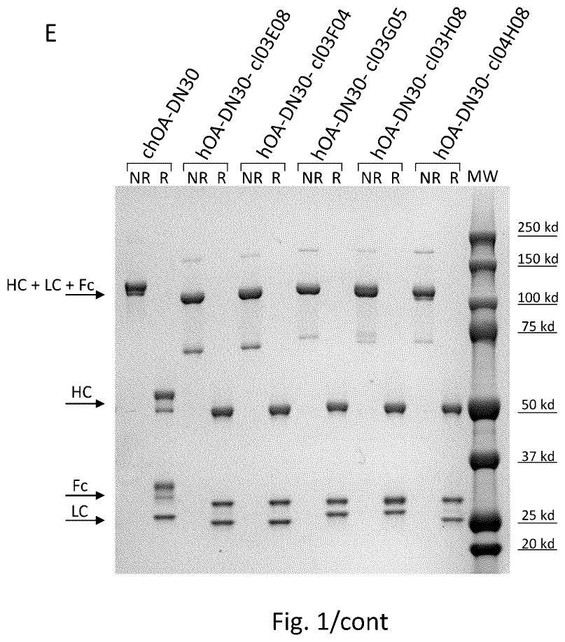

To format the chimeric and the humanized antibodies into the One-Arm single chain form, specific amino acid modifications have been inserted in the CH3 region to produce the ‘knob into hole’ structure^{28,29}; one CH3 domain carries the knob mutation: ³⁸⁹T>W, the other CH3 domain carries the hole mutations: ³⁸⁹T->S; ³⁹¹L->A; ⁴³⁸Y>V. CDNAs encoding for the chimeric or humanized light chains, the chimeric or humanized heavy chains (mutated in the CH3 domain) and the human Fc domain (mutated in the CH3 domain) were cloned into expression plasmids and then expressed into eukaryotic cells. Assembled One-Arm antibodies were purified from cell culture supernatants by affinity chromatography and gel filtration. FIG. 1 shows a schematic drawing of the DN30 derivatives and the SDS-Page separation under not reducing and reducing condition of the purified recombinant OA-DN30 molecules.

### DN30 One-Arm Antibodies Recognize Met With High Affinity

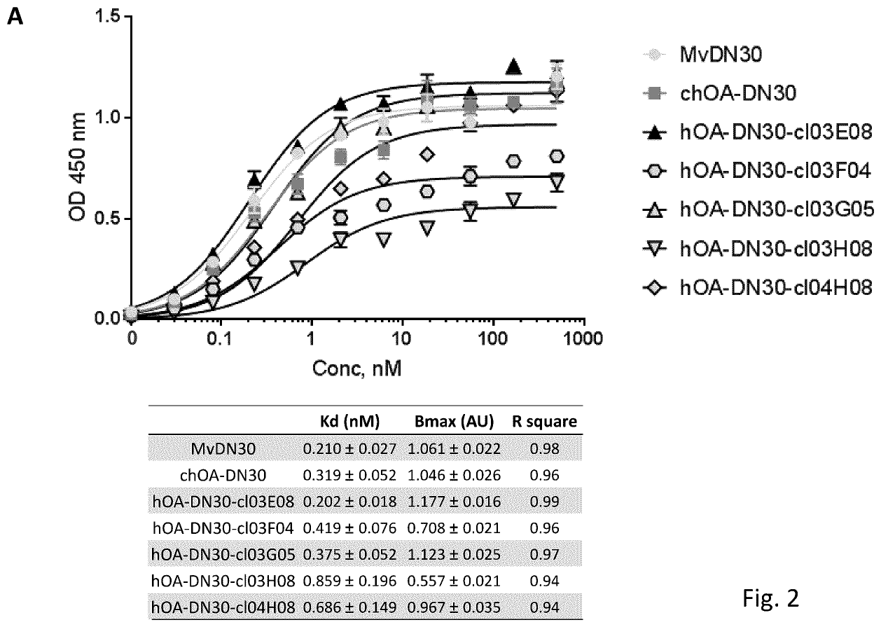

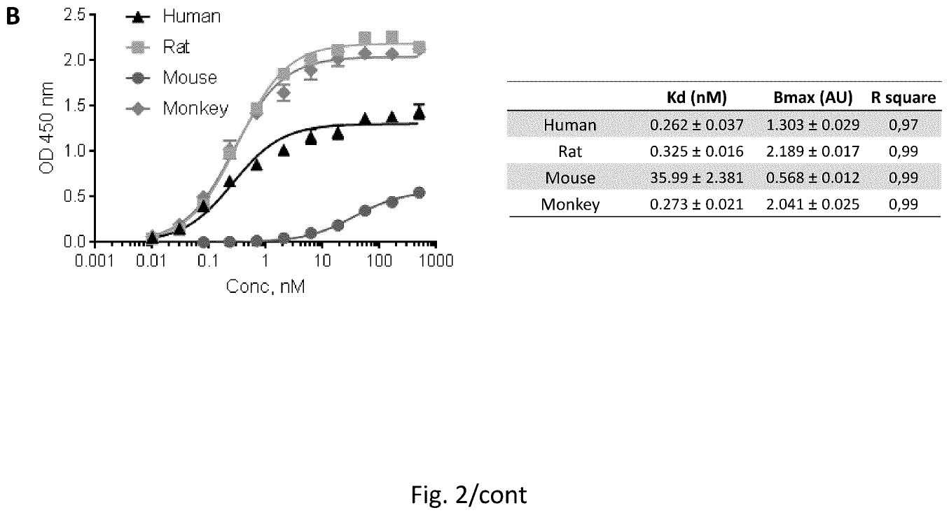

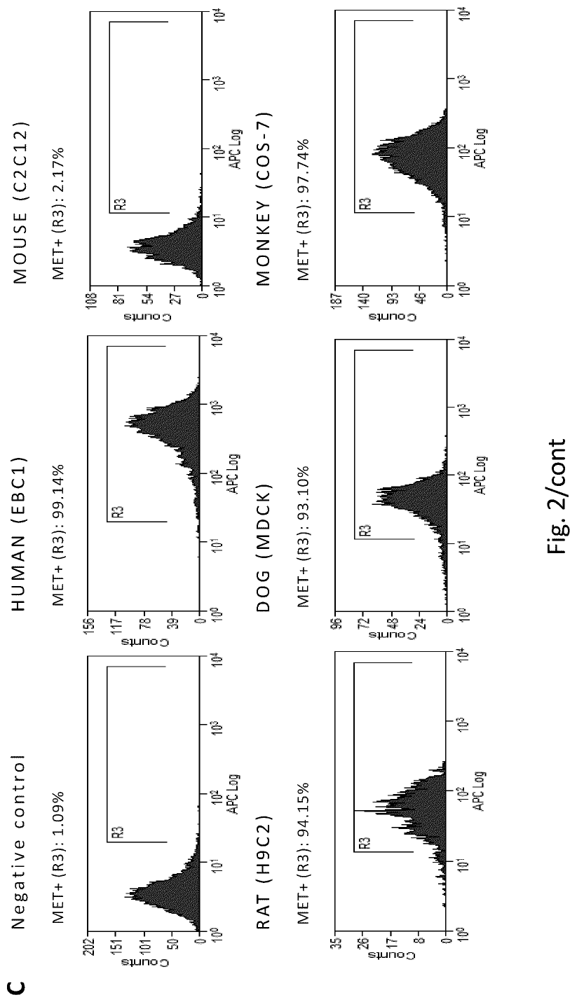

Purified One-Arm forms of the chimeric or humanized DN30 antibodies were analysed for binding to the Met receptor by an ELISA assay. As reference, the chimeric DN30 Fab (MvDN30) 12 was included in the analysis. The assay was assembled to include Met-Fc in solid phase and the DN30 derivatives in liquid phase. Binding was revealed using anti-human K chain antibodies. Data showed that both the chimeric and the humanized antibodies bind to Met, the hOA-DN30-cl03E08 showing the highest affinity (FIG. 2A).

Analysis of hOA-DN30-cl03E08 Cross-Reactivity With Non-Human Met Receptors

The present inventors analysed the species cross reactivity of the hOA-DN30-cl03E08 performing: (i) an ELISA assay with purified Met extracellular domains of human, mouse, rat, and monkey origin (FIG. 2B); (ii) a flow-cytometer analysis of antibody binding to surface Met expressed by cells of human, mouse, rat, dog and monkey origin (FIG. 2C). Results of the experiments showed that hOA-DN30-cl03E08 binds Met of human, rat, dog, monkey origin, while the interaction with mouse Met was very weak.

### DN30 One-Arm Antibodies Do Not Exert Agonistic Properties

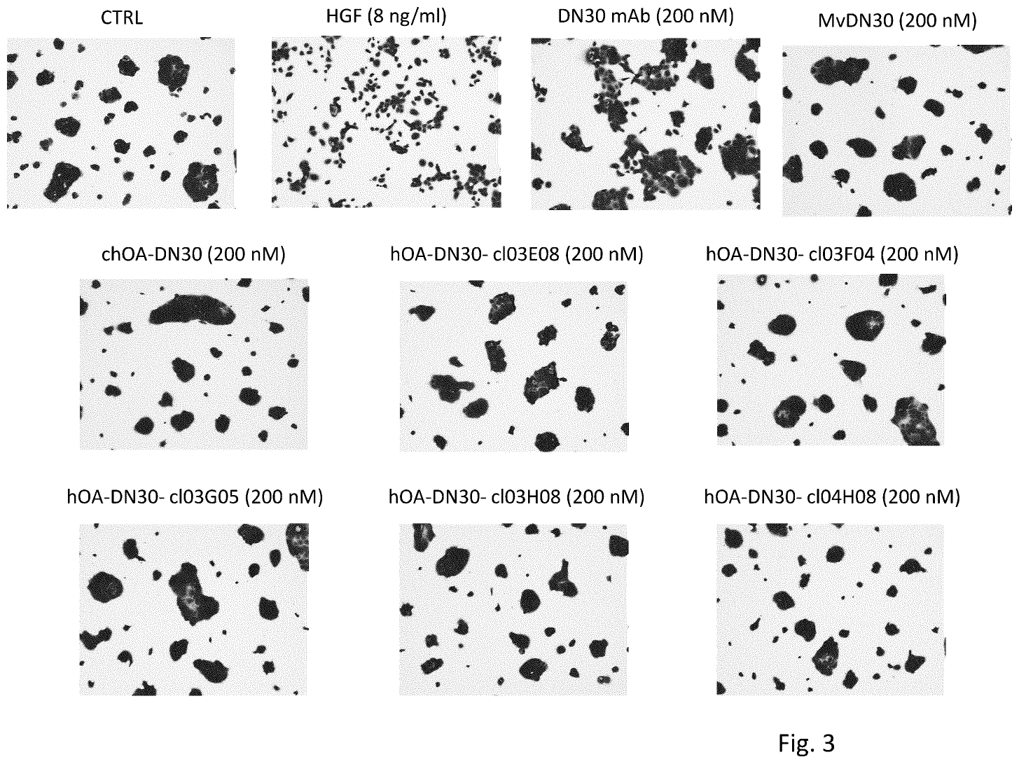

The present inventors tested whether the new DN30 derivatives could display Met agonistic activity in a highly sensitive assay, the scatter assay. HPAF-II human pancreatic carcinoma cells, which represent a standard system for determining cell motility in response to HGF stimulation, were stimulated for 24 hrs with increasing concentrations of the antibody. As a positive control, HGF and bivalent DN30 mAb were included in the assay. While cells stimulated with the positive controls were clearly scattered, the phenotype of the cells treated with all the monovalent DN30 derivatives (MvDN30, chOA-DN30, and hOA-DN30 antibodies) was indistinguishable from the untreated ones (FIG. 3).

hOA-DN30 Potently Induces Met ‘Shedding’

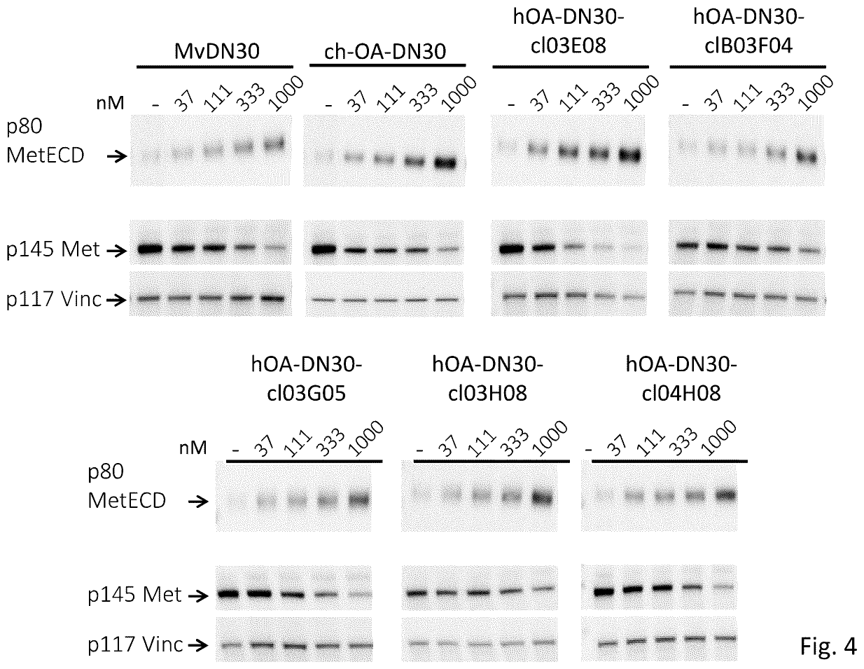

The present inventors also investigated whether hOA-DN30 maintained the ability to promote receptor shedding and downregulation. A549 cells were incubated with increasing concentrations of choA-DN30 and hOA-DN30 antibodies. As a reference, MvDN30 was included in the assay. After 48 hours, Met ectodomains released in the conditioned medium were scored by immunoblotting using a monoclonal antibody directed against the extracellular portion of Met. Total cellular levels of Met were also determined on cell lysates using the same antibody. This analysis revealed that all the OA-DN30 antibodies induced Met shedding, resulting in the release of soluble Met ectodomains in the extracellular space and physically removing Met from the cell surface (FIG. 4). Unexpectedly, the activity of the hOA-DN30-cl03E08 was superior as compared with all the other DN30 derivatives included in the test.

hOA-DN30-cl03E08 Strongly Inhibits Met Phosphorylation

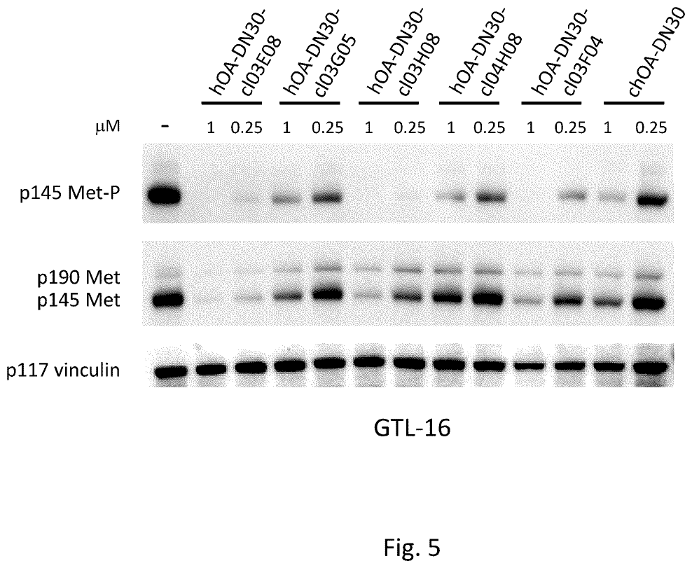

The present inventors investigated if the choA-DN30 and the hOA-DN30 Met antibodies inhibited phosphorylation in cells derived from human carcinomas, i.e. GTL-16. These cells have a constitutive active Met as the consequence of receptor over-expression due to gene amplification. Met activation was determined by immunoblotting with anti-phosphoMet antibodies. As shown in FIG. 5, all the molecules impaired Met phosphorylation/activation, with a dose-response modality. hOA-DN30-cl03E08, inducing the strongest impairment of Met levels in the cells, was the DN30 derivative more active.

hOA-DN30-cl03E08 Powerfully Inhibits MET-Addicted Cell Growth

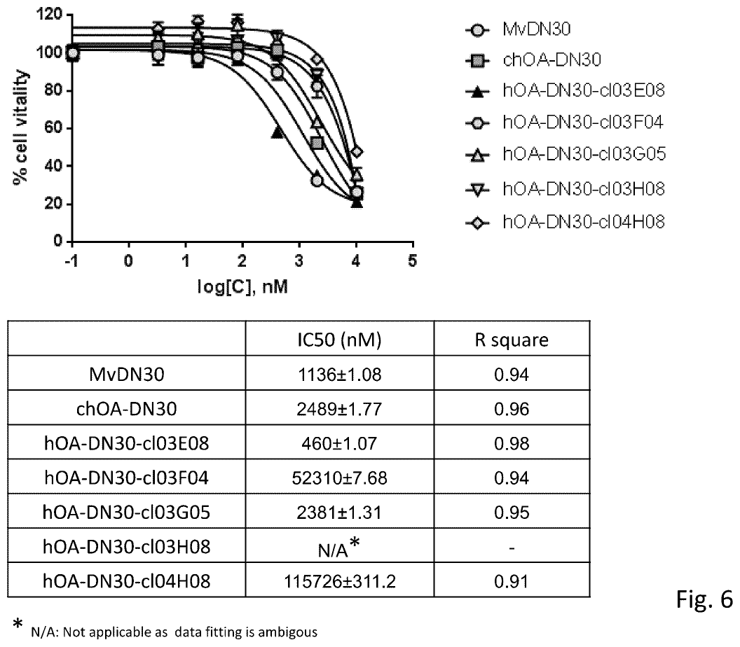

Cell growth (viability) can be impaired by a Met-inhibitor only in MET-addicted cells, which rely on Met-signalling for proliferation/survival. To test the activity of the DN30 derivatives, exponentially growing GTL-16 cells (MET-addicted human gastric carcinoma cells) were incubated with increasing concentrations of chOA-DN30 or hOA-DN30 antibodies. MvDN30 was included in the assay as a positive control. After 72 hours, cell viability was determined using a luminescence-based ATP assay. All the DN30 derivatives inhibited MET-addicted cell growth in a dose-dependent manner (FIG. 6). hOA-DN30-cl03E08 showed the highest inhibition, being the IC₅₀ 2.5 folds lower as compared to MvDN30, 5.4 folds lower as compared to the choA-DN30, and at least 5.2 folds lower as compared to the other hOA-DN30 antibodies (see table in FIG. 6).

hOA-DN30-cl03E08 is Highly Effective in Blocking Proliferation and in Inducing Cytotoxicity in Met-Addicted Cells

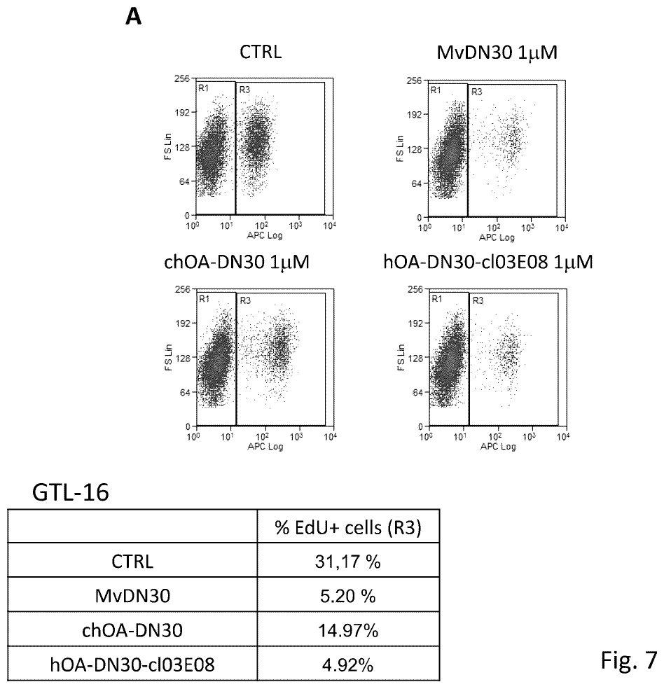

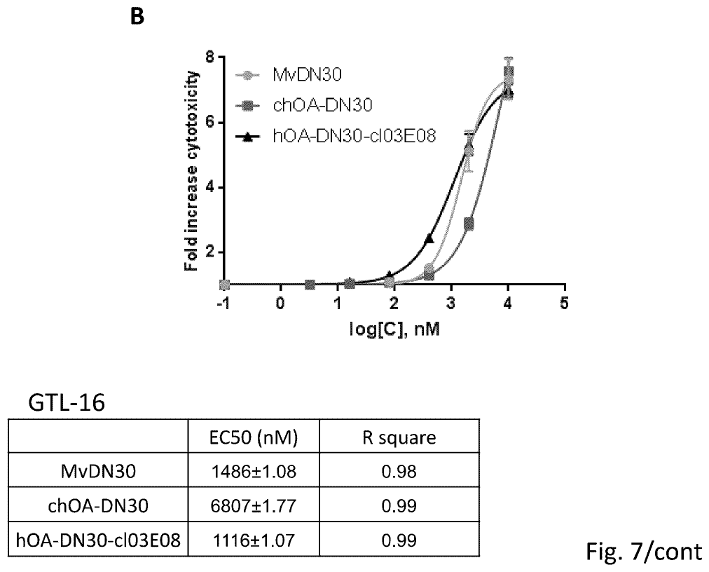

To further analyze the mechanisms underlining the inhibition of MET-addicted cell growth exerted by the DN30 derivatives, the inventors evaluated cell proliferation and cell cytotoxicity, comparing MvDN30, chOA-DN30, and hOA-DN30-cl03E08. GTL-16 cells were incubated with a single dose (1 μM) of the DN30 derivatives and cell proliferation was assessed by measuring the incorporation of EdU™—a thymidine analogous-by the cells during the S phase of the cell cycle, with the ClickIT Edu flow cytometry assay. MvDN30 was included in the assay as a positive control. This analysis revealed that DN30 derivatives affected proliferation, as the percentage of treated cells in S-phase was dramatically reduced as compared with control. In particular, the most effective molecule was hOA-DN30-cl03E08, where only 4.9% of cells were still proliferating (84.2% reduction versus control). The proliferating cells in the population treated with choA-DN30 were the 14.97% (52% reduction versus control) (FIG. 7A).

To test if DN30 derivatives induced only a block of proliferation or if they caused also cytotoxicity, exponentially growing MET-addicted GTL-16 cells were incubated with increasing concentrations of choA-DN30 or hOA-DN30-cl03E08. MvDN30 was included in the assay as a positive control. After 72 hours, cytotoxicity was determined adding CellTox™ Green dye to the cells and measuring the fluorescence of the cells that is directly proportional to the number of dead cells. All the DN30 derivatives induced cytotoxicity in GTL-16 cells in a dose-dependent manner (FIG. 7B). hOA-DN30-cl03E08 showed the highest induction, being the EC₅₀ 1.3 and 6.1 folds lower as compared to MvDN30 and chOA-DN30, respectively.

hOA-DN30-cl03E08 Inhibits HGF-Induced Cell Motility, Alone and in Combination With DecoyMet

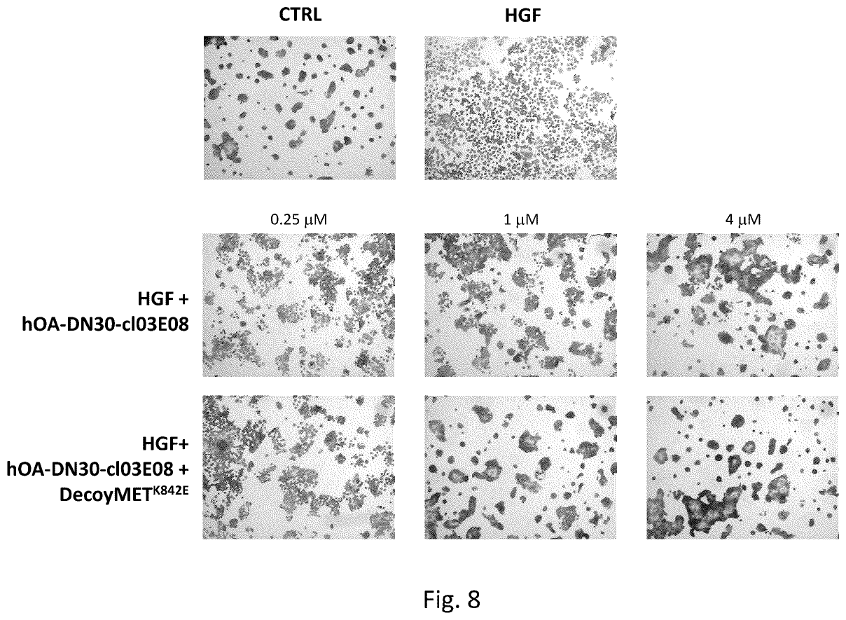

The present inventors tested the ability of hoA-DN30-cl03E08 to inhibit HGF-dependent cell motility of HPAF-II human pancreatic carcinoma cells. Cells were incubated or not with HGF and treated with increasing dose of hOA-DN30-cl03E08. After 24hrs, cell colonies were stained and analysed. In this assay, hOA-DN30-cl03E08 strongly reduced HGF-dependent cell scattering (FIG. 8).

In the above cellular system it has been reported that a more effective inhibition of the HGF-induced biological response can be achieved by blocking concomitantly the ligand (HGF) and the receptor (Met)²⁷. The present inventors thus tested if hOA-DN30-cl03E08 in combination with DecoyMet^{K842E} induced a higher therapeutic response. DecoyMet^{K842E} is a recombinant soluble receptor encompassing the whole extracellular region of Met carrying a mutation that abolishes the interaction with DN30; it binds HGF with high affinity and inhibits ligand-driven biological activities by sponging and neutralizing HGF and by forming heterodimers with intact Met receptors still present on the cell surface, rendering them inactive. With equimolar amount of the two molecules in combination, the effective dose completely reverting the scattered phenotype was 4 times lower than the dose of hOA-DN30-cl03E08 alone able to eliciting a similar cell phenotype (FIG. 8).

hOA-DN30-cl03E08 Inhibit HGF-Induced Cell Invasion, Alone and in Combination With DecoyMet

The present inventors tested the ability of hoA-DN30-cl03E08 to inhibit HGF-dependent cell invasion of HPAF-II cells. Cells were seeded in the upper chambers of transwell filters coated with matrigel™ in serum free medium with hOA-DN30-cl03E08 (0.5 μM) alone or in combination with DecoyMet^{K842E} (1 μM). The lower chambers were filled with medium containing HGF (25 ng/ml). After 24 hrs, cells migrating on the lower part of the filter were evaluated by staining and microscope observation. In this assay, hOA-DN30-cl03E08 strongly reduced HGF-dependent cell invasion; the cell response was almost abolished by the combination hOA-DN30-cl03E08 and DecoyMet^{K842E} (FIG. 9).

hOA-DN30-cl03E08 Revokes Interferon-Gamma Induction of PD-L1 Expression

Programmed cell death ligand 1 (PD-L1) and programmed cell death receptor 1 (PD-1) are key modulatory molecules, known as immune-checkpoints, that play a central role at the interface between immune response and tumor microenvironment³⁰. They may significantly impair the ability of the immune system to control tumor progression. The expression of PD-L1 by tumor cells is inducible and interferon gamma (IFNgamma) is the most potent inducer³¹.

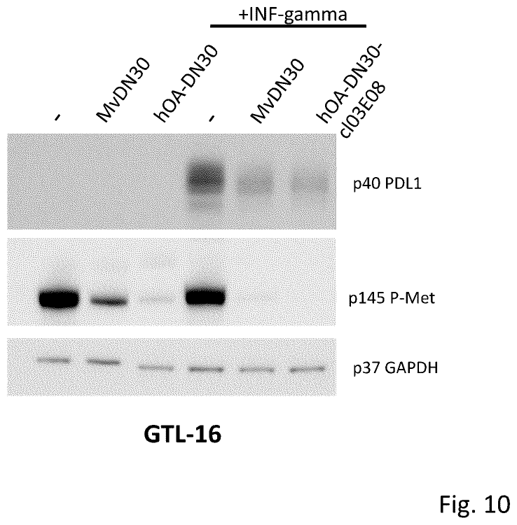

The present inventors analyzed if inhibition of Met by hOA-DN30-cl03E08 could modulate the IFNgamma pathway and consequently PD-L1 regulation (FIG. 10). MET-addicted GTL-16 cells were treated with INFgamma for 48 hrs and analyzed for IFNgamma-inducible PD-L1expression. In an unstimulated condition, PD-L1 was not detectable, while upon exposure to IFNgamma, it was consistently up-regulated. Treatment with hOA-DN30-C103E08 for 48 hrs significantly impaired the up-regulation of PD-L1 induced by IFNgamma.

hOA-DN30-cl03E08 Shows a Favorable Pharmacokinetic Profile In Vivo

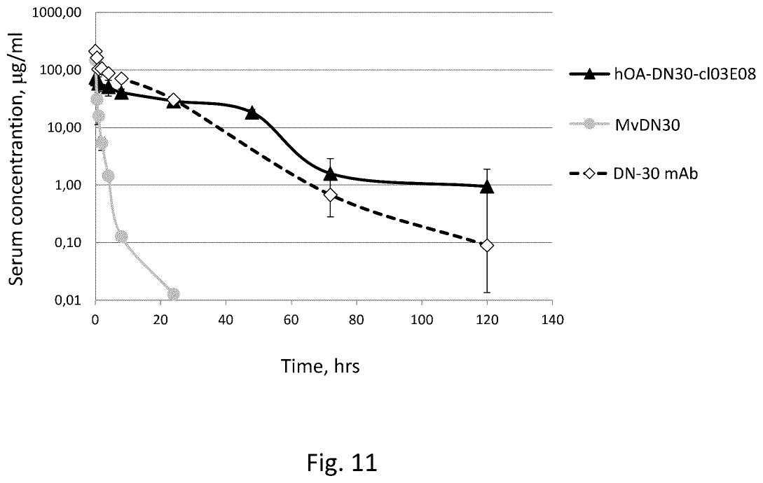

The present inventors studied the pharmacokinetic properties of hOA-DN30-C103E08, in comparison with MvDN30 and the chimeric DN30 mAb. A single dose of the above mentioned molecules were delivered by intravenous injection to immunodeficient mice. Peripheral blood from the treated mice was collected at different time points after the delivery. The circulating concentrations of the studied molecules were determined by ELISA performed on serum samples. hOA-DN30-cl03E08 circulating levels were comparable to the ones measured for the mAb, and always higher than MvDN30 (FIG. 11).

hOA-DN30-cl03E08 Impairs Growth of MET-Addicted Tumors In Vivo

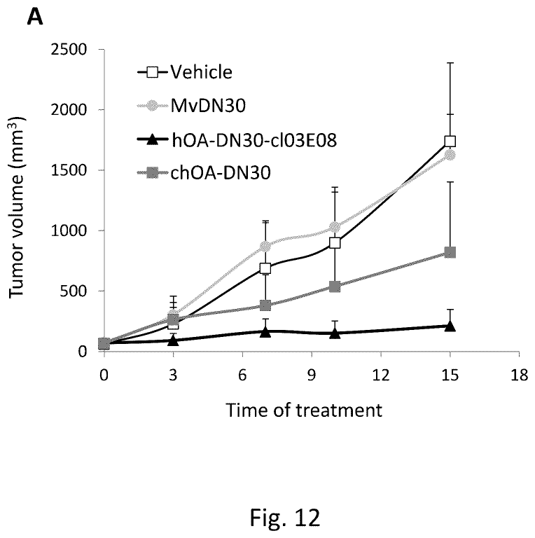

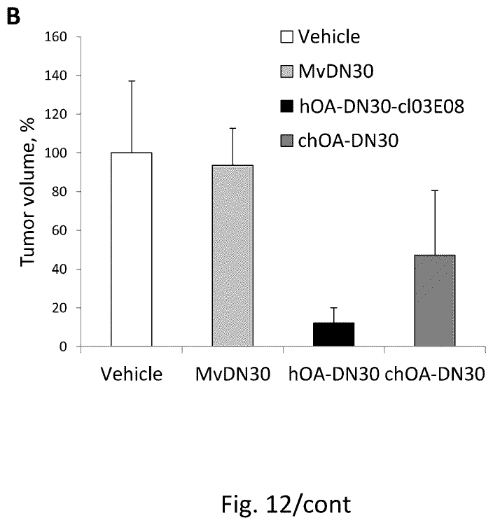

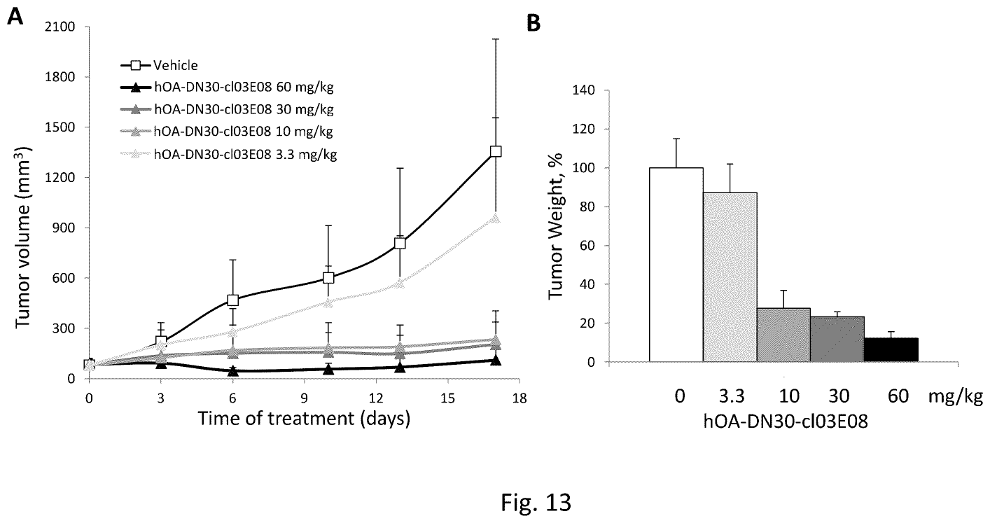

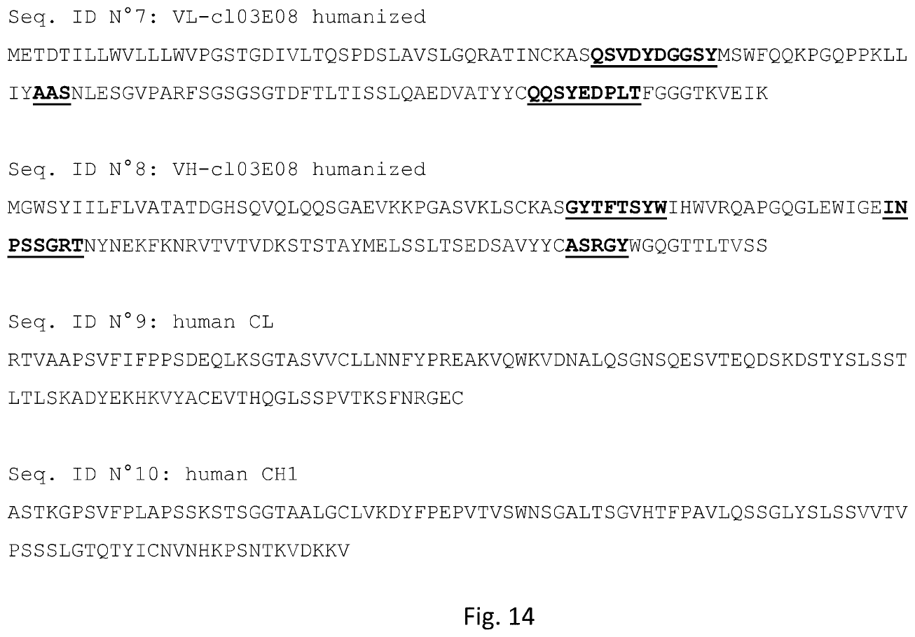

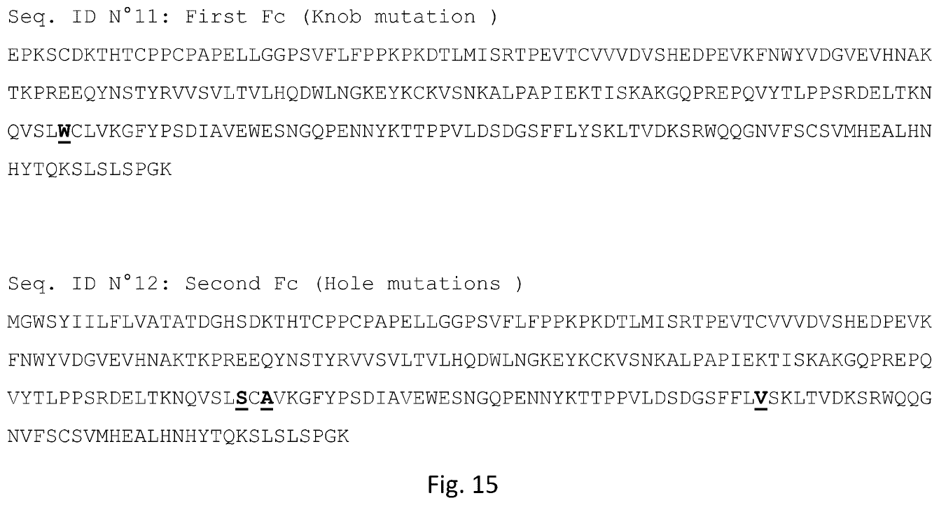

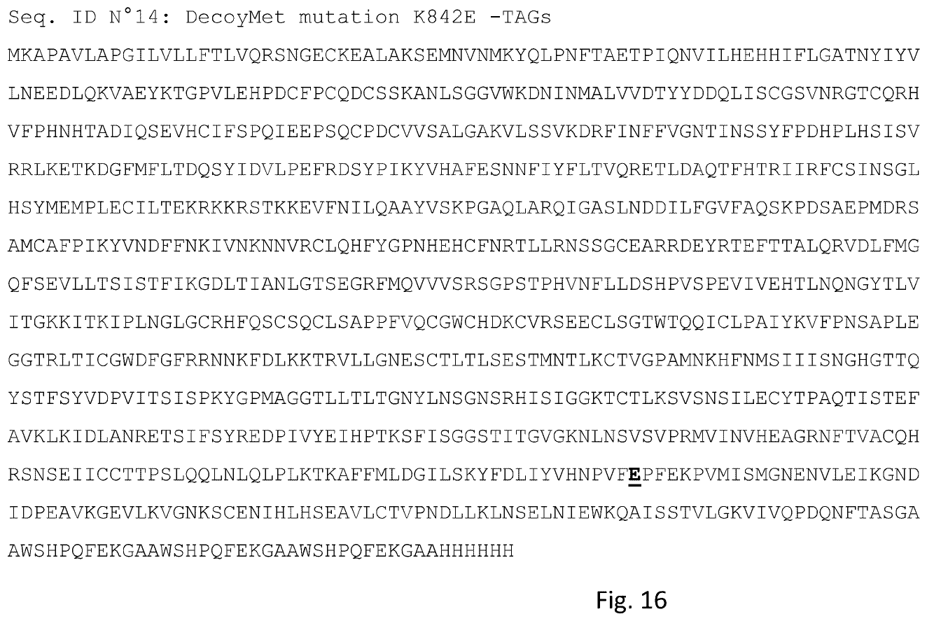

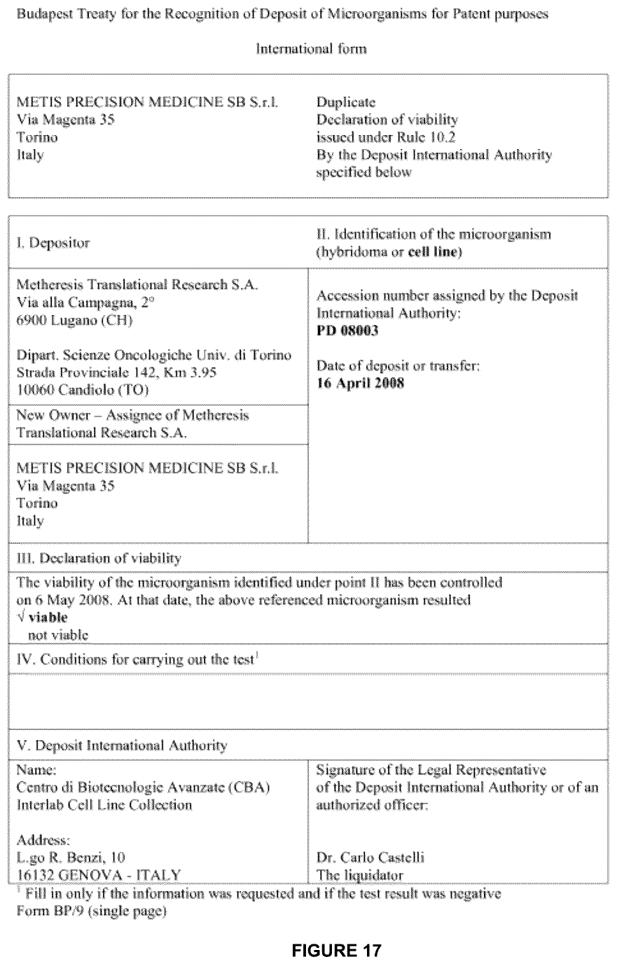

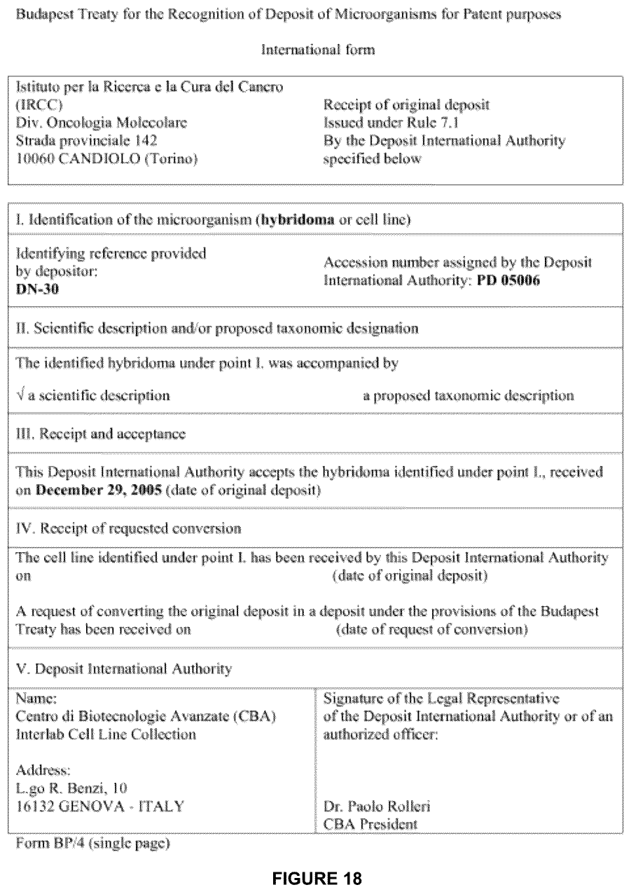

The ability of DN30-derivatives to inhibit tumor growth in vivo was tested on a MET-addicted model. 1×10⁶ GTL-16 cells were sub-cutaneously injected in the flank of NOD-SCID mice. After 1 week, mice carrying palpable tumors were randomized in 4 homogenous treatment groups: Vehicle (n=6), MvDN30 (n=6), hOA-DN30-cl03E08 (n=5), chOA-DN30 (n=5). Antibodies were delivered by intravenous injection twice/week at 30 mg/Kg; to administer the same amount of molecules, MvDN30 was delivered following the same schedule at 15 mg/kg, as the molecular weight of the One-arm antibodies are double with respect to MvDN30 (i.e. around 100 KDa for OA-DN30 formats and around 50 KDa for MvDN30). MvDN30 was not effective—as expected from its short plasma half-life—while both the One-Arm derivatives inhibited tumor growth (FIG. 12A) (P=0.0007 and 0.05 for hOA-DN30-cl03E08 and for choA-DN30 respectively versus vehicle). Surprisingly hOA-DN30-cl03E08 was exceedingly more effective than choA-DN30: the treated masses were, in the case of hOA-DN30-cl03E08, 87.8% and, in the case of chOA-DN30, 52.9% smaller than controls (FIG. 12B) (P=0.009 for hOA-DN30-cl03E08 versus chOA-DN30). The present inventors further analyzed the inhibitory activity of the hOA-DN30-cl03E08 performing a dose response experiment. Maintaining the experimental conditions described above, mice were randomized in 5 homogenous groups and treated by i.v. injection three times/week with different doses of hOA-DN30-cl03E08. Doses of 10, 30, and 60 mg/kg were highly effective in blocking tumor growth, while treatment with 3.3 mg/kg was not statistically different from controls (FIG. 13).

### Material and Methods

### Cell Culture

A549 human lung adenocarcinoma cells, HPAF-II human pancreatic adenocarcinoma cells, C2C12 mouse muscle myoblasts, H9C2 (2-1) rat cardiac myoblasts, MDCK dog kidney cells, and Cos-7 monkey kidney cells were obtained from ATCC™/LGC Standards S.r.l. (Sesto San Giovanni, Italy); GTL-16 cell line is a clone derived from MKN-45 cells (human gastric carcinoma cells available by the Japanese Collection of Research Bioresources, Osaka,

Japan) that differs in MET gene copy number from the parental cell line³². GTL-16 cell line is available by Advanced Biotechnology Center (ABC), Interlab Cell Line Collection (ICLC) Italy, with accession number ICLC PD 08003. EBC-1, human lung cancer, were from Japanese Collection of Research Bioresources). DN30 hybridoma is available by Advanced Biotechnology Center (ABC), Interlab Cell Line Collection (ICLC) Italy, with accession number ICLC PD 05006.

All the cells were cultured as suggested by the supplier. All cell cultures were tested for mycoplasma contamination.

### Generation of the Chimeric and Human One-Arm Form of the DN30 Antibody

For the chimeric DN30 antibody: three separate CDNAs encoding respectively the light chain (VL-CL), the heavy chain (VH-CH1-CH2-CH3), and the Fc domain (CH2-CH3) have been generated by gene synthesis. Variable regions (mouse sequences) were from the DN30 antibody (SEQ ID: 15 and 16); constant regions of human origin derived from human immunoglobulins, in particular the light chain includes the human kappa type constant domain (GenBank™ sequence ID #: DI165992), and the heavy chain includes the human IgG1 constant domains (GenBank sequence ID #: DJ392898). At the C-terminus of the encoding sequences, the heavy chain includes a His-TAG (SEQ ID: 38, 39) and the Fc includes a strep-TAG (SEQ ID: 40, 41).

For the humanized DN30 antibody: Humanization by germ line screening of the DN30 mouse antibody has been done by Fair Journey Biologics using a phage display library approach. They started from the identification of deviating FR (frame) residues and the analysis of the closest human V germline, chosen from germlines with identical canonical fold combination for CDR1-CDR2, using available tools and databases: abYsistool from Dr. Andrew Martin at UCL plus, bioc.uzh.ch/plueckthun/antibody/and mrc-lmb.cam.ac.uk/vbase/. Upon generation of phage display libraries-in which randomization of the VH and VL region has been performed-four affinity-driven phage display rounds of selection have been done. At the end of the process, five different DN30 humanized variants with the highest human identity and homology were selected, namely clones 03E08, 03G05, 03F04, 03H08 and 04H08. Variable regions were linked through classical molecular biology techniques to the constant domain sequences derived from human immunoglobulins, i.e. human kappa light chain and IgG1 heavy chain constant domains.

For formatting the chimeric and the humanized antibodies into the One-Arm form (chOA-DN30 and hOA-DN30 antibodies): specific amino acid modifications have been inserted in the CH3 region to produce the ‘knob into hole’ structure^{28,29}. ‘Knobs-into-holes’ was demonstrated to be an effective design strategy for enhancing the formation of heterodimers over homodimers. The strategy is based on the domain interface remodeling, introducing sterically complementary mutations in the CH3 region of the antibody Fc fragment, the most extensive site of protein-protein interaction between the H chains of human IgG molecules. One chain, the VH-CH1-CH2-CH3 polypeptide, includes the ³⁸⁹T>W (Knob mutation), in which a small amino acid is substituted by a larger one; conversely, the other chain, the CH2-CH3 polypeptide, includes the replacement of large residues with smaller ones: ³⁸⁹T>S; ³⁹¹L>A; ⁴³⁸Y->V (Hole mutations). These mutations were selected upon optimization by phage display technology³³, in which residues 389, 391 and 438, that are in proximity to the knob on the partner CH3 domain, were randomly substituted. Knobs-into-holes engineering facilitates the assembly of a heterodimer, including the three different amino acid chains (VL-CL+VH-CH1-CH2-CH3+CH2-CH3) in place of homodimers i.e. the bona fide antibody (VL-CL+VH-CH1-CH2-CH3)₂ or the Fc domain only (CH2-CH3)₂.

At the N-terminal, the CH2-CH3 polypeptide includes a sequence corresponding to a truncated human Hinge region. The deletion is necessary to exclude the cysteine at position 233 according to the EU numbering scheme of Kabat that is involved in the formation of an inter-chain disulfide bond with the cysteine at the C-terminal position of the light chain constant domain (CL). Moreover, to allow the CH2-CH3 polypeptide to enter the RER/Golgi pathway of protein synthesis—as light (VL-CL) and heavy chains (VH-CH1-CH2-CH3) of the antibody do—at the N-terminus, before the truncated Hinge region, a sequence corresponding to the signal peptide of the DN30 Heavy chain is included.

The chimeric anti-Met antibody fragment (chOA-DN30) disclosed herein is made of:

(i) a light chain variable domain having the amino acid sequence set forth in SEQ ID No.: 15;

(ii) a heavy chain variable domain having the amino acid sequence set forth in SEQ ID No.: 16;

(iii) a human light chain constant (CL) domain having the amino acid sequence set forth in SEQ ID No.: 9;

(iv) a human heavy chain constant CH1 domain having the amino acid sequence set forth in SEQ ID No.: 10;

(v) a first human Fc polypeptide having the amino acid sequence set forth in SEQ ID No.: 11; and

(vi) a second human Fc polypeptide having the amino acid sequence set forth in SEQ ID No.: 12.

The humanized anti-Met antibody fragment (hOA-DN30-cl03E08) object of the present disclosure is made of:

(i) a humanized light chain variable domain having the amino acid sequence set forth in SEQ ID No.: 7;

(ii) a humanized heavy chain variable domain having the amino acid sequence set forth in SEQ ID No.: 8;

(iii) a human light chain constant (CL) domain having the amino acid sequence set forth in SEQ ID No.: 9;

(iv) a human heavy chain constant CH1 domain having the amino acid sequence set forth in SEQ ID No.: 10;

(v) a first human Fc polypeptide having the amino acid sequence set forth in SEQ ID No.: 11; and

(vi) a second human Fc polypeptide having the amino acid sequence set forth in SEQ ID No.: 12.

The humanized anti-Met antibody fragment (hOA-DN30-cl03G05) is made of:

(i) a humanized light chain variable domain having the amino acid sequence set forth in SEQ ID No.: 17;

(ii) a humanized heavy chain variable domain having the amino acid sequence set forth in SEQ ID No.: 18;

(iii) a human light chain constant (CL) domain having the amino acid sequence set forth in SEQ ID No.: 9;

(iv) a human heavy chain constant CH1 domain having the amino acid sequence set forth in SEQ ID No.: 10;

(v) a first human Fc polypeptide having the amino acid sequence set forth in SEQ ID No.: 11; and

(vi) a second human Fc polypeptide having the amino acid sequence set forth in SEQ ID No.: 12.

The humanized anti-Met antibody fragment (hOA-DN30-cl03F04) is made of:

(i) a humanized light chain variable domain having the amino acid sequence set forth in SEQ ID No.: 19;

(ii) a humanized heavy chain variable domain having the amino acid sequence set forth in SEQ ID No.: 20;

(iii) a human light chain constant (CL) domain having the amino acid sequence set forth in SEQ ID No.: 9;

(iv) a human heavy chain constant CH1 domain having the amino acid sequence set forth in SEQ ID No.: 10;

(v) a first human Fc polypeptide having the amino acid sequence set forth in SEQ ID No.: 11; and

(vi) a second human Fc polypeptide having the amino acid sequence set forth in SEQ ID No.: 12.

The humanized anti-Met antibody fragment (hOA-DN30-cl03H08) object of the present disclosure is made of:

(i) a humanized light chain variable domain having the amino acid sequence set forth in SEQ ID No.: 21;

(ii) a humanized heavy chain variable domain having the amino acid sequence set forth in SEQ ID No.: 22;

(iii) a human light chain constant (CL) domain having the amino acid sequence set forth in SEQ ID No.: 9;

(iv) a human heavy chain constant CH1 domain having the amino acid sequence set forth in SEQ ID No.: 10;

(v) a first human Fc polypeptide having the amino acid sequence set forth in SEQ ID No.: 11; and

(vi) a second human Fc polypeptide having the amino acid sequence set forth in SEQ ID No.: 12.

The humanized anti-Met antibody fragment (hOA-DN30-cl04H08) object of the present disclosure is made of:

(i) a humanized light chain variable domain having the amino acid sequence set forth in SEQ ID No.: 23;

(ii) a humanized heavy chain variable domain having the amino acid sequence set forth in SEQ ID No.: 24;

(iii) a human light chain constant (CL) domain having the amino acid sequence set forth in SEQ ID No.: 9;

(iv) a human heavy chain constant CH1 domain having the amino acid sequence set forth in SEQ ID No.: 10;

(v) a first human Fc polypeptide having the amino acid sequence set forth in SEQ ID No.: 11; and

(vi) a second human Fc polypeptide having the amino acid sequence set forth in SEQ ID No.: 12.

### Expression and Purification of Chimeric and Human One Arm Antibodies

The cDNAs encoding the above described light chains (nucleotide sequence set forth in SEQ ID No.: 25, 26, 27, 28, 29 for the humanized light chains and SEQ ID No.: 30 for the chimeric light chain), heavy chains (nucleotide sequence set forth in SEQ ID No.: 31, 32, 33, 34, 35 for the humanized heavy chain and SEQ ID No.: 36 for the chimeric heavy chain) and Fc (nucleotide sequence set forth in SEQ ID No.: 37) were cloned into commonly available expression vectors (e.g. pcDNA3.1 plasmids cat. #V79020 Invitrogen Corporation, Camarillo, CA) and used to transfect ExpiCHO-S™ cells (cat. #: A29127, ThermoFisher Scientific). The vector ratio was optimized to maximize the output of the desired 97.5 kDa heterodimer (the OA-Ab) and to minimize the presence of any 150 kDa bivalent homodimer (bona fide Mab). The optimal ratio was: 1:1:2 respectively for Light chain, Heavy chain full size (“knob” vector) and Fc (“hole” vector). This condition resulted in the least amount of homodimer present, with the heterodimer being approximately 85% of the total protein. Transient expression was powered ExpiFectamine™ CHO transfection reagent (cat. #A29130, Thermo Fisher Scientific). Supernatants were harvested after 9 days of production. The OA-DN30 antibodies were purified using a Hitrap MabSelect Sure column (cat. #GE29-0491-04, Sigma Aldrich) on an AKTA Pure 25 chromatography system. The OA-DN30 antibodies were subsequently eluted using 0.1 M citrate buffer at pH 3.0. Eluted fractions were collected and neutralized with 1M Tris-HCl pH 9.0 (at a ratio of 0.15 ml per ml of eluted protein). The OA-DN30 antibodies were further purified on a 1× PBS (pH 7.4)-equilibrated Superdex 200 26/600 size exclusion column (cat. #GE28-9893-36, Sigma Aldrich) on a GE AKTA Prime Liquid Chromatography System (GE Healthcare Life Sciences), where fractions containing the OA-Ab were separated from homodimeric fractions (mAb or Fc). Preparative SEC fractions containing the OA-DN30 antibodies were pooled and analyzed by SDS-PAGE to ascertain the purity. The pooled fractions were also tested for endotoxin levels. The final pooled fractions from the preparative SEC purifications, showing the absence of any bivalent homodimer, and the presence of a small amount (<5%) of a smaller ˜70 kDa band, which corresponds to the Knob-Knob Fc homodimer.

### Generation, Expression, and Purification of Mutated Met Ectodomain

CDNA sequence of human MET ectodomain (DecoyMet) carrying a single amino acid substitution was synthetically generated starting from the human Met ectodomain sequence as disclosed in GenBank™ at deposit number X54559, and using the QuickChange II™ Site-Directed Mutagenesis Kit (cat. #200524 Agilent Technologies, Santa Clara, CA), following the instruction of the manufacturer. The procedure requires the design of sense and antisense oligonucleotides, that include the desired point mutation. The following oligos has been employed:

mutation K842E:
(SEQ ID No.: 42)
sn. 5′-gtacataatcctgtgtttgagccttttgaaaagccagtg-3′;
(SEQ ID No.: 43)
as. 5′-cactggcttttcaaaaggctcaaacacaggattatgtac-3′.

Engineered soluble receptor was produced by transient transfection of HEK-293T cells with pcDNA3.1 plasmids (cat. #V79020 Invitrogen Corporation, Camarillo, CA) expressing cDNA encoding for DecoyMet mutant. Transfected cells were starved for three days and cell culture supernatants containing the soluble receptor were collected. Purification of the recombinant proteins was done by affinity chromatography using HisTrap™ HP columns (cat. #17524701 GE Healthcare, Freiburg, Germany) according to manufacturer's instructions. Large-scale protein production and purification was performed by U-Protein Express BV (Utrecht, The Netherlands).

The variant of human DecoyMet (DecoyMETK842E) has the amino acid sequences set forth in SEQ ID No.: 14.

### Analysis of the Purified Proteins by SDS-PAGE+Blue Coomassie

Purified OA-DN30 antibodies (1 μg) were separated into a 4-12% acrylamide gradient gel by SDS-PAGE in the presence of in the absence of □-mercapto ethanol following standard methods. Molecular weight markers (cat. #1610374, Bio-Rad) were included in the analysis. Polypeptides separated into the gel were revealed by Gel Code Blue Stain™ reagent (cat. #24590, Thermo Fisher Scientific).

### ELISA Binding Assays

For analysis of the interaction between Met and the DN30 derivatives, purified Met-Fc chimeras (cat #. 358-MT-100 R&D Systems, 100 ng/well) were immobilized on ELISA plates. After saturation with 0.5% BSA incubated 1 hr at 37° C., increasing concentrations of the antibodies (MvDN30, chOA-DN30 or hOA-DN30 antibodies, 0-500 nM prepared in PBS-BSA 0.5%-Tween™ 0.1%)) were added in liquid phase. Binding was revealed using HRP-conjugated anti-human k chain antibody (cat. #A7164, Sigma-Aldrich) followed by incubation with TMB (cat. #T8665, Sigma Aldrich). Colorimetric assay was quantified by the multi-label plate reader VICTOR-X4 (Perkin Elmer Instruments INC., Whaltman, MA). Data were analyzed and fit using Prism software (GraphPad).

For ELISA binding analysis of hOA-DN30-cl03E08 (liquid phase) to Met-Fc chimera (solid phase) derived from different species, Met of human (cat #. 358-MT-100 R&D Systems), murine (cat. #50622-M02H, Sino Biological), rat (cat. #80004-R02H, Sino Biological), and monkey (cat. #90304-C02H, Sino Biological) origin were included in the assay. The procedure was same as the one described above.

### Flow-Cytometer Binding Analysis

For hOA-DN30-cl03E08 binding to Met expressed at the surface of cells, 2×10⁵ EBC-1, C2C12, H9C2, MDCK, or Cos-7 cells were treated with StemPro™ Accutase™ Cell Dissociation Reagent (cat. #A1110501, Thermo Fisher Scientific) collected and incubated for 1 hour at 4°° C. in PBS-2% FCS. staining Antibody was performed by incubating the cells for 30 min at 4° C. with 10 μg/ml of HOA-DN30-cl03E08 in PBS-2% BSA. After 6 washes with PBS-2% BSA, cells were incubated with anti-human IgG-APC (cat. #109-606-088 Jackson Immuno Research) diluted 1:100 for 30 min al 4° C. and then washed to removed unbound antibodies. Cells were co-stained with DAPI (3μl of 1 μg/ml working solution, Sigma-Aldrich, cat. # 10236276001) for 5 min at 4° C. and analyzed for Met expression by Summit 4.3 software (Dako). The fluorescence signal derived from the isotype control (anti human APC IgG) was set as a threshold (0<MFI<101). Cells were considered positive for Met expression if the Mean Fluorescence Intensity (MFI) was higher than the threshold (MFI>101).

### Analysis of Met Shedding

Sub-confluent A549 monolayers were washed twice with PBS and then incubated in serum-free medium with increasing concentrations (37, 111, 333, 1000 nM) of DN-30 derivatives. After 48 hours, conditioned medium was collected and cells were lysed with Laemmli Buffer. Met protein levels were determined in 15 μg of total cellular proteins by Western blot using anti-Met antibodies (3D4, cat. #08-1366 Invitrogen Corporation); as a loading control, filters were probed also with anti-vinculin antibodies (clone hVIN-1, cat. #V9131 Sigma Life Science). Met ectodomain levels were determined in 15 μl of cell culture supernatant by Western blotting using an anti-human HGFR/Met antibody directed against the extracellular domain of the receptor (cat. #AF276, R&D Systems). Anti-mouse IgG1 HRP-conjugated secondary antibodies (cat. #JI115035003, Jackson ImmunoResearch) and the ECL System (cat. #W1015, Promega,) were used for protein detection. Western Blot bands were quantified using ImageJ software.

### MET Phosphorylation Assays

Serum-starved GTL-16 cells were incubated for 24 h with DN30 derivatives (1000 or 250 nM). Total cellular lysates were analyzed by Western blot using the following primary antibodies: anti-Met phospho-Tyr1234/1235 (D26, cat. #3077 Cell Signaling Technology); anti-Met (3D4, cat. #08-1366 Invitrogen Corporation); and anti-vinculin (clone hVIN-1, cat. #V9131 Sigma Life Science). Anti-mouse IgG1 and anti-rabbit IgG (cat. #JI111035003) HRP-conjugated secondary antibodies and the ECL System were used for protein detection. Western Blot bands were quantified using ImageJ software.

### In Vitro Biological Assays

For cell scattering assays, HPAF-II cells (8000/well) were seeded in 96-well plates in complete culture medium. To analyze the agonistic activity of the antibodies, after 24 hrs, HGF (8 ng/ml, positive control, cat. #294-HG-025, R&D Systems) or the antibodies (DN30 mAb, MvDN30, chOA-DN30, hOA-DN30 antibodies, all at the concentration of 200nM), were added to the culture medium. To analyze the inhibitory activity, increasing concentrations (0-4 μM) of hOA-DN30-cl03E08, alone or in 1:1 combination with DecoyMet^{K842E}, were added. After an additional time of 24 hrs, cells were stimulated with 6.25 ng/ml HGF for 24 hrs. Cells were then fixed with 11% glutaraldehyde (cat. #340855 Sigma-Aldrich), stained with 0.1% Crystal Violet (cat. #C3886 Sigma-Aldrich) and analyzed by microscope observation (Microscope Leica DM2000). Images were captured with QICAM Fast 1394 color digital Camera (QImaging).

For cell invasion assays, HPAF-II cells (1.5×10⁵/well) were suspended in serum-free culture medium in the presence of 0.5 μM hOA-DN30-cl03E08 alone or in combination with 1 μM DecoyMet^{K842E}, and seeded on the upper compartment of transwell chambers pre-coated with 30 μg/well of Matrigel™ Matrix (cat. #354234, Corning Inc.). Culture medium supplemented with 2% FBS and 6.25 ng/ml HGF was added to the lower compartment of the chambers. After 24 hrs, cells on the upper side of the transwell filters were mechanically removed, while cells migrated through the membrane were fixed with 11% glutaraldehyde and stained with 0.1% Crystal Violet. Cell invasion was quantified with Image-J software.

For vitality assay, GTL-16 cells were seeded 2000 cells/well in a 96 well plate in 10% FBS culture medium. After 24 hrs, medium was replaced with a fresh one with 10% FBS plus the molecules to be tested (increasing concentrations-from 0 to 10 μM). Cell viability was evaluated after 72 hrs using the CellTiter-Glo (cat. # G7573 Promega Corp), according to the manufacturer's instructions. Chemo-luminescence was detected with VICTOR X4.

For proliferation assay, 350000 GTL-16 cells were plated in 6 well plates in 10% FCS medium. After 24 hrs, cells were treated with a fixed concentration (1 μM) of DN30 derivatives for further 48 hrs. Then, 10 μM EdU (cat. #A10044, Thermo Fisher) was added to the culture medium for a further 2 hrs. The % of cells in S phase was determined by cytofluorimeter analysis following the procedure of the Click-iT™ EdU Alexa Fluor™ 488 Flow Cytometry Assay (cat. #C10425, Thermo Fisher).

For cytotoxicity assay, 2000 GTL-16 cells were plated in 96 well plates in 10% FCS medium. After 24 hrs, cells were treated with increasing concentrations of the DN30 derivatives for a further 48 hrs. Cell cytotoxicity was evaluated by Cell-Tox™ Green cytotoxicity assay (cat. #G8741, Promega) following manufacturer's instructions.

### PD-L1 Expression Analysis

Sub-confluent GT1-16 cells were treated with 50 ng/ml of IFN□-1b (Miltenyi Biotec, cat. #130-096-484) for 48 hours (replaced every 24 hours) in combination with 250nM hOA-DN30-cl03E08. Monolayers were then lysed in Laemmli buffer and 45 μg of total proteins were subjected to 8% SDS-PAGE gel. Proteins were transferred from the gel onto iBlot™ Transfer nitrocellulose membranes (Life technologies, cat. #IB23001) following standard methods. PD-L1 expression was detected by anti-PD-L1 antibody (E1L3N, cat. #13684, Cell Signaling Technology). P-Met levels were checked by anti-MET phospho-Tyr^{1234/1235} (D26) antibodies. As loading control, filters were probed with anti-GAPDH (D4C6R, cat. #97166 Cell Signaling Technology). Secondary HRP-conjugated goat anti-mouse IgG (cat. #JI115035003) or anti-rabbit IgG (cat. #JI111035144) (both from Jackson ImmunoResearch) and the ECL System were used for protein detection.

### In Vivo Experiments

All animal procedures were performed according to protocols approved by Ethical Committee for animal experimentation of the Fondazione Piemontese per la Ricerca sul Cancro and by Italian Ministry of Health. NOD-SCID mice were purchased from Charles River (Calco, Italy).

For pharmacokinetic analysis NOD-SCID mice were injected intravenously with a single dose (100 μg) of hOA-DN30-cl03E08. Peripheral blood was collected at different time points: 10′, 30′, 1 hr, 4 hrs, 8 hrs, 24 hrs, 48 hrs, 72 hrs, 120 hrs. Serum concentrations of the therapeutic molecules were measured by ELISA as described the binding assay section, above in interpolating the absorbance values of the samples on the linear part of a standard curve obtained by serial dilutions of the different purified molecules. Each time point was the average value of 4 mice.

For tumor growth analysis 1×10⁶ GTL-16 cells were inoculated in the flank of NOD-SCID mice. Tumor growth was monitored by caliper measurement twice weekly. Tumor volume was calculated using the formula: V=4/3 □ (x/2) (y/2) (z/2), where x, y and z are height, width and depth of the tumor mass. When the tumors reached a volume of 70-100 mm³ (approximately one week after cell injection) mice were randomized in homogeneous groups. In one case, groups were generated (PBS-vehicle, MvDN30, chOA-DN30, hOA-DN30-cl03E08; all the antibodies 30 mg/kg, 2 times per week); in the other case, 5 groups were generated (PBS-vehicle, hOA-DN30-cl03E08 60 mg/kg, 30 mg/kg, 10 mg/kg, 3.3 mg/kg; all the doses 3 times per week). Molecules were administered by intravenous injection. At the end of the experiments (15 or 17 days of treatment) mice were sacrificed, tumors were excised and weighted.

### Statistical Analysis

Average and standard deviation (SD) were calculated using Microsoft Office Excel 2010 software (Microsoft Corporation). To calculate Ka values, data from ELISA assays were analyzed and fitted according to nonlinear regression, one site binding hyperbola curve, using GraphPad Prism software (GraphPad Software). To calculate IC₅₀ and EC₅₀ values, data were analyzed and fitted according to nonlinear regression, sigmoidal dose-response curve, using GraphPad Prism software. All experiments were repeated at least two times. FIGS. show one representative experiment.

### REFERENCES

ADDIN EN. REFLIST 1. Comoglio P M, Trusolino L, Boccaccio C. Known and novel roles of the MET oncogene in cancer: a coherent approach to targeted therapy. Nat Rev Cancer 2018; 18 (6): 341-358; doi 10.1038/s41568-018-0002-y.
2. Kong-Beltran M, Seshagiri S, Zha J, Zhu W, Bhawe K, Mendoza N et al. Somatic mutations lead to an oncogenic deletion of met in lung cancer. Cancer Res 2006; 66 (1): 283-289; doi 10.1158/0008-5472.CAN-05-2749.
3. Gandino L, Longati P, Medico E, Prat M, Comoglio P M. Phosphorylation of serine 985 negatively regulates the hepatocyte growth factor receptor kinase. J Biol Chem 1994; 269 (3): 1815-1820.
4. Petrelli A, Gilestro G F, Lanzardo S, Comoglio P M, Migone N, Giordano S. The endophilin-CIN85-Cbl complex mediates ligand-dependent downregulation of c-Met. Nature 2002; 416 (6877): 187-190; doi 10.1038/416187a.
5. Trusolino L, Bertotti A, Comoglio P M. MET signalling: principles and functions in development, organ regeneration and cancer. Nat Rev Mol Cell Biol 2010; 11 (12): 834-848; doi 10.1038/nrm3012.
6. Prat M, Crepaldi T, Pennacchietti S, Bussolino F, Comoglio P M. Agonistic monoclonal antibodies against the Met receptor dissect the biological responses to HGF. J Cell Sci 1998; 111 (Pt 2): 237-247.
7. Pacchiana G, Chiriaco C, Stella M C, Petronzelli F, De Santis R, Galluzzo M et al. Monovalency unleashes the full therapeutic potential of the DN-30 anti-Met antibody. J Biol Chem 2010; 285 (46): 36149-36157; e-pub ahead of print 2010 Sep. 10; doi 10.1074/jbc.M110.134031.
8. Cignetto S, Modica C, Chiriaco C, Fontani L, Milla P, Michieli P et al. Dual Constant Domain-Fab: A novel strategy to improve half-life and potency of a Met therapeutic antibody. Mol Oncol 2016; 10 (6): 938-948; e-pub ahead of print 2016 Mar. 28; doi 10.1016/j.molonc.2016.03.004.
9. Wang Q, Yang S, Wang K, Sun S Y. MET inhibitors for targeted therapy of EGFR TKI-resistant lung cancer. J Hematol Oncol 2019; 12 (1): 63; e-pub ahead of print 2019 Jun. 21; doi 10.1186/s13045-019-0759-9.
10. Schelter F, Kobuch J, Moss M L, Becherer J D, Comoglio P M, Boccaccio C et al. A disintegrin and metalloproteinase-10 (ADAM-10) mediates DN30 antibody-induced shedding of the met surface receptor. J Biol Chem 2010; 285 (34): 26335-26340; doi 10.1074/jbc.M110.106435.
11. Foveau B, Ancot F, Leroy C, Petrelli A, Reiss K, Vingtdeux V et al. Down-regulation of the met receptor tyrosine kinase by presenilin-dependent regulated intramembrane proteolysis. Mol Biol Cell 2009; 20 (9): 2495-2507; doi 10.1091/mbc.E08-09-0969.
12. Vigna E, Chiriaco C, Cignetto S, Fontani L, Basilico C, Petronzelli F et al. Inhibition of ligand-independent constitutive activation of the Met oncogenic receptor by the engineered chemically-modified antibody DN30. Mol Oncol 2015; 9 (9): 1760-1772; doi 10.1016/j.molonc.2015.05.007.
13. Jones P T, Dear P H, Foote J, Neuberger M S, Winter G. Replacing the complementarity-determining regions in a human antibody with those from a mouse. Nature 1986; 321 (6069): 522-525; doi 10.1038/321522a0.
14. Riechmann L, Clark M, Waldmann H, Winter G. Reshaping human antibodies for therapy. Nature 1988; 332 (6162): 323-327; doi 10.1038/332323a0.
15. Presta L G. Antibody engineering. Curr Opin Biotechnol 1992; 3 (4): 394-398.
16. Vaswani S K, Hamilton R G. Humanized antibodies as potential therapeutic drugs. Ann Allergy Asthma Immunol 1998; 81 (2): 105-115; quiz 115-106, 119; doi 10.1016/S1081-1206 (10) 62794-9.
17. Hurle M R, Gross M. Protein engineering techniques for antibody humanization. Curr Opin Biotechnol 1994; 5 (4): 428-433.
18. Harris W J. Production of humanized monoclonal antibodies for in vivo imaging and therapy. Biochem Soc Trans 1995; 23 (4): 1035-1038.
19. Bloom J W, Madanat M S, Marriott D, Wong T, Chan S Y. Intrachain disulfide bond in the core hinge region of human IgG4. Protein Sci 1997; 6 (2): 407-415; doi 10.1002/pro. 5560060217.
20. Humphreys D P, Chapman A P, Reeks D G, Lang V, Stephens P E. Formation of dimeric Fabs in Escherichia coli: effect of hinge size and isotype, presence of interchain disulphide bond, Fab′ expression levels, tail piece sequences and growth conditions. J Immunol Methods 1997; 209 (2): 193-202.
21. Ferracini R, Longati P, Naldini L, Vigna E, Comoglio P M. Identification of the major autophosphorylation site of the Met/hepatocyte growth factor receptor tyrosine kinase. J Biol Chem 1991; 266 (29): 19558-19564.
22. Ponzetto C, Bardelli A, Zhen Z, Maina F, dalla Zonca P, Giordano S et al. A multifunctional docking site mediates signaling and transformation by the hepatocyte growth factor/scatter factor receptor family. Cell 1994; 77 (2): 261-271.
23. Gherardi E, Love C A, Esnouf R M, Jones E Y. The sema domain. Curr Opin Struct Biol 2004; 14 (6): 669-678; doi 10.1016/j.sbi.2004.10.010.
24. Bork P, Doerks T, Springer T A, Snel B. Domains in plexins: links to integrins and transcription factors. Trends Biochem Sci 1999; 24 (7): 261-263.
25. Merchant M, Ma X, Maun H R, Zheng Z, Peng J, Romero M et al. Monovalent antibody design and mechanism of action of onartuzumab, a MET antagonist with anti-tumor activity as a therapeutic agent. Proc Natl Acad Sci U S A 2013; 110 (32): E2987-2996; doi 10.1073/pnas.1302725110.
26. Makabe K, Nakanishi T, Tsumoto K, Tanaka Y, Kondo H, Umetsu M et al. Thermodynamic consequences of mutations in vernier zone residues of a humanized anti-human epidermal growth factor receptor murine antibody, 528. J Biol Chem 2008; 283 (2): 1156-1166; e-pub ahead of print 2007 Oct. 17; doi 10.1074/jbc.M706190200.
27. Basilico C, Modica C, Maione F, Vigna E, Comoglio P M. Targeting the MET oncogene by concomitant inhibition of receptor and ligand via an antibody-“decoy” strategy. Int J Cancer 2018; e-pub ahead of print 2018 Apr. 25; doi 10.1002/ijc.31550.
28. Ridgway J B, Presta L G, Carter P. ‘Knobs-into-holes’ engineering of antibody CH3 domains for heavy chain heterodimerization. Protein Eng 1996; 9 (7): 617-621.
29. Zhu Z, Presta L G, Zapata G, Carter P. Remodeling domain interfaces to enhance heterodimer formation. Protein Sci 1997; 6 (4): 781-788; doi 10.1002/pro.5560060404.
30. Zou W, Chen L. Inhibitory B7-family molecules in the tumour microenvironment. Nat Rev Immunol 2008; 8 (6): 467-477; doi 10.1038/nri2326.
31. Garcia-Diaz A, Shin D S, Moreno B H, Saco J, Escuin-Ordinas H, Rodriguez G A et al. Interferon Receptor Signaling Pathways Regulating PD-L1 and PD-L2 Expression. Cell Rep 2017; 19 (6): 1189-1201; doi 10.1016/j.celrep.2017.04.031.
32. Rege-Cambrin G, Scaravaglio P, Carozzi F, Giordano S, Ponzetto C, Comoglio P M et al. Karyotypic analysis of gastric carcinoma cell lines carrying an amplified c-met oncogene. Cancer Genet Cytogenet 1992; 64 (2): 170-173.
33. Atwell S, Ridgway J B, Wells J A, Carter P. Stable heterodimers from remodeling the domain interface of a homodimer using a phage display library. J Mol Biol 1997; 270 (1): 26-35; doi 10.1006/jmbi.1997.1116.

1. An anti-Met antibody fragment comprising a single antigen binding arm and an Fc region (that increases stability of said antibody fragment compared to a Fab molecule comprising said antigen binding arm), wherein the Fc region comprises a complex of a first and a second Fc polypeptide, wherein the antibody fragment comprises:
(i) a first polypeptide comprising one humanized light chain variable (VL) domain, and one human light chain constant (CL) domain, wherein the humanized VL domain is fused to the human CL domain in the N-to C-term direction, and wherein the humanized VL domain contains three complementary determining regions (CDRs) having amino acid sequences as set forth in SEQ ID No.: 1, 2 and 3, and wherein the humanized VL domain has an amino acid sequence as set forth in SEQ ID No.: 7;
(ii) a second polypeptide comprising one humanized heavy chain variable (VH) domain, one human heavy chain constant CH1 domain and the first Fc polypeptide, wherein the first Fc polypeptide comprises one hinge region, one human constant CH2 domain and one human CH3 constant domain, wherein the humanized VH domain is fused to the human CH1 domain, that is fused to the human hinge region, that is fused to the human CH2 domain, that is fused to the human CH3 domain in the N- to C-term direction, and wherein the humanized VH domain contains three complementary determining regions (CDRs) having amino acid sequences as set forth in SEQ ID No.: 4, 5 and 6, and wherein the humanized VH domain has an amino acid sequence as set forth in SEQ ID No.: 8; and
(iii) a third polypeptide comprising the second human Fc polypeptide, wherein the second human Fc polypeptide comprises one human hinge region, one human constant CH2 domain and one human CH3 constant domain, wherein the human hinge region is fused to the CH2 domain that is fused to the human CH3 domain in the N-to C-term direction, wherein the human hinge region is truncated at the N-terminus.

2. The anti-Met antibody fragment according to claim 1, wherein the human CL domain is a human light kappa type domain.

3. The anti-Met antibody fragment according to claim 1 or claim 2, wherein the human hinge region and the human constant domains CH1, CH2 and CH3 are from a human IgG1.

4. The anti-Met antibody fragment according to any one of claims 1 to 3, wherein the two Fc polypeptides are linked through intermolecular disulfide bonds at the hinge region.

5. The anti-Met antibody fragment according to any one of claims 1 to 4, wherein the first Fc polypeptide and the second Fc polypeptide meet at an interface, and one between the first and the second Fc polypeptide comprises a knob at the interface, and the other between the first and the second Fc polypeptide comprises a hole at the interface, wherein the knob is positionable into the hole.

6. The anti-Met antibody fragment according to claim 5, wherein one between the first and the second Fc polypeptide comprises a mutated CH3 constant domain, wherein the mutated CH3 constant domain carries an amino acid mutation at position 389, wherein the original amino acid at position 389 has been mutated to import an amino acid having a larger side chain volume than the original amino acid; and wherein the other between the first and the second Fc polypeptide comprises a mutated CH3 constant domain, wherein the mutated CH3 constant domain carries three amino acid mutations at positions 389, 391 and 438, wherein the original amino acids have been mutated to import amino acids having smaller side chains volume than the original amino acids, wherein the amino acid numbering is according to the EU numbering scheme of Kabat.

7. The anti-Met antibody fragment according to claim 6, wherein the original amino acids at positions 389, 391 and 438 are threonine, leucine and tyrosine respectively; and wherein in one between the first and the second Fc polypeptide the threonine in position 389 has been mutated to tryptophan; and wherein in the other between the first and the second Fc polypeptide the threonine at position 389 has been mutated to serine, the leucine at position 391 has been mutated to alanine and the tyrosine at position 438 has been mutated to valine.

8. The anti-Met fragment according to any one of claims 1 to 7, wherein the human CL domain has an amino acid sequence as set forth in SEQ ID No.: 9 and the human CH1 domain has an amino acid sequence as set forth in SEQ ID No.: 10.

9. The anti-Met antibody fragment according to any one of claims 1 to 8, wherein the first human Fc polypeptide has an amino acid sequence as set forth in SEQ ID No.: 11, and the second human Fc polypeptide has an amino acid sequence as set forth in SEQ ID No.: 12.

10. The anti-Met antibody fragment according to any one of claims 1 to 9, wherein the anti-Met antibody fragment when bound to Met induces shedding of an extracellular domain of Met.

11. Isolated nucleic acid encoding the anti-Met antibody fragment of any of claims 1 to 10.

12. A composition comprising two or more recombinant nucleic acids which collectively encode the anti-Met antibody fragment of any of claims 1 to 10.

13. A product comprising, in a single bottle or in two bottles, (a) an anti-Met antibody fragment according to any one of claims 1 to 10, and a pharmaceutically acceptable vehicle, and (b) an extracellular portion of human Met and a pharmaceutically acceptable vehicle, wherein the extracellular portion of human Met is capable of binding to Hepatocyte Growth Factor (HGF) in a stable manner and contains at least one amino acid mutation within the epitope recognized by the anti-Met antibody fragment to prevent binding of the anti-Met antibody fragment thereto.

14. The product according to claim 13, wherein the extracellular portion of human Met contains SEMA, PSI, IPT-1, IPT-2, IPT-3 and IPT-4 domains.

15. The product according to claim 13 or claim 14, wherein the extracellular portion of human Met has an amino acid sequence as set forth in SEQ ID No.: 13, wherein at least one of the amino acids between position 797 and position 875 of SEQ ID No.: 13 is mutated in order to prevent binding of the anti-Met antibody fragment thereto.

16. The product according to any one of claims 13 to 15, wherein the extracellular portion of human Met has an amino acid sequence as set forth in SEQ ID No.: 14.

17. An anti-Met antibody fragment according to any one of claims 1 to 10 or a product according to any one of claims 13 to 16 for use in the treatment of a patient suffering from a tumor and/or metastasis, wherein the patient carries genetic alterations of the MET gene.

18. A product according to any one of claims 13 to 16 for use in the treatment of a patient suffering from a tumor and/or metastasis, wherein the patient carries a wild-type MET gene.


SEQUENCE LISTING (43 sequences)

SEQ ID NO: 1 - protein, 10 aa, synthetic construct
  source 1..10: note = VL-CDR1
QSVDYDGGSY

SEQ ID NO: 2 - skipped

SEQ ID NO: 3 - protein, 9 aa, synthetic construct
  source 1..9: note = VL-CDR3
QQSYEDPLT

SEQ ID NO: 4 - protein, 8 aa, synthetic construct
  source 1..8: note = VH-CDR1
GYTFTSYW

SEQ ID NO: 5 - protein, 8 aa, synthetic construct
  source 1..8: note = VH-CDR2
INPSSGRT

SEQ ID NO: 6 - protein, 5 aa, synthetic construct
  source 1..5: note = VH-CDR3
ASRGY

SEQ ID NO: 7 - protein, 131 aa, synthetic construct
  source 1..131: note = humanized VL-cl03E08
METDTILLWVLLLWVPGSTGDIVLTQSPDSLAVSLGQRATINCKASQSVDYDGGSYMSWFQQKPGQPPKLLIYAASNLESGVPARFSGSGSGTDFTLTISSLQAEDVATYYCQQSYEDPLTFGGGTKVEIK

SEQ ID NO: 8 - protein, 131 aa, synthetic construct
  source 1..131: note = humanized VH-cl03E08
MGWSYIILFLVATATDGHSQVQLQQSGAEVKKPGASVKLSCKASGYTFTSYWIHWVRQAPGQGLEWIGEINPSSGRTNYNEKFKNRVTVTVDKSTSTAYMELSSLTSEDSAVYYCASRGYWGQGTTLTVSS

SEQ ID NO: 9 - protein, 107 aa, Homo sapiens [sequence omitted]

SEQ ID NO: 10 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 11 - protein, 232 aa, synthetic construct
  source 1..232: note = First Fc (Knob mutation)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 246 aa, synthetic construct
  source 1..246: note = Second Fc (Hole mutations)
MGWSYIILFLVATATDGHSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 976 aa, synthetic construct
  source 1..976: note = Decoy Met wt-TAGs
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTASGAAWSHPQFEKGAAWSHPQFEKGAAWSHPQFEKGAAHHHHHH

SEQ ID NO: 14 - protein, 976 aa, synthetic construct
  source 1..976: note = Decoy Met mutation K842E-TAGs
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFEPFEKPVMISMGNENVLEIKGNDIDPEAVKGEVLKVGNKSCENIHLHSEAVLCTVPNDLLKLNSELNIEWKQAISSTVLGKVIVQPDQNFTASGAAWSHPQFEKGAAWSHPQFEKGAAWSHPQFEKGAAHHHHHH

SEQ ID NO: 15 - protein, 131 aa, synthetic construct
  source 1..131: note = murine VL
METDTILLWVLLLWVPGSTGDIVLTQSPASLAVSLGQRATISCKASQSVDYDGGSYMSWFQQRPGQPPKLLISAASNLESGIPARFSGSGSGTDFTLNIHPVEEEDVATYYCQQSYEDPLTFGAGTKVEIK

SEQ ID NO: 16 - protein, 131 aa, synthetic construct
  source 1..131: note = murine VH
MGWSYIILFLVATATDGHSQVQLQQPGTELVKPGASVKLSCKASGYTFTSYWIHWVKQRPGQGLEWIGEINPSSGRTNYNEKFKNKVTVTVDKSSTTAYMQLSNLTSEDSAVYYCASRGYWGQGTTLTVSS

SEQ ID NO: 17 - protein, 131 aa, synthetic construct
  source 1..131: note = humanized VL-cl03G05
METDTILLWVLLLWVPGSTGDIVLTQSPASLAVSLGERATISCKASQSVDYDGGSYMSWFQQRPGQPPKLLIYAASNLESGVPDRFSGSGSGTDFTLNISPLEAEDVAVYYCQQSYEDPLTFGGGTKVEIK

SEQ ID NO: 18 - protein, 131 aa, synthetic construct
  source 1..131: note = humanized VH-cl03G05
MGWSYIILFLVATATDGHSQVQLVQPGTEVKKPGASVKLSCKASGYTFTSYWIHWVRQAPGQGLEWMGEINPSSGRTNYNEKFKNRVTITADKSTSTAYMQLSSLRSEDTAVYYCASRGYWGQGTTVTVSS

SEQ ID NO: 19 - protein, 131 aa, synthetic construct
  source 1..131: note = humanized VL-cl03F04
METDTILLWVLLLWVPGSTGDIVMTQSPDSLAVSLGERATINCKASQSVDYDGGSYMSWYQQKPGQPPKLLISAASNLESGVPDRFSGSGSGTDFTLTIHSLQAEDVAVYYCQQSYEDPLTFGAGTKVEIK

SEQ ID NO: 20 - protein, 131 aa, synthetic construct
  source 1..131: note = humanized VH-cl03F04
MGWSYIILFLVATATDGHSQVQLQQPGAEVKKPGSSVKLSCKASGYTFTSYWIHWVKQAPGQGLEWIGEINPSSGRTNYNEKFKNKVTITVDKSTSTAYMELSNLRSEDSAVYYCASRGYWGQGTTLTVSS

SEQ ID NO: 21 - protein, 131 aa, synthetic construct
  source 1..131: note = humanized VL-cl03H08
METDTILLWVLLLWVPGSTGDIVLTQSPASLAVSLGERATISCKASQSVDYDGGSYMSWYQQKPGQPPKLLIYAASNLESGVPDRFSGSGSGTDFTLNISSVQEEDVATYYCQQSYEDPLTFGAGTKVEIK

SEQ ID NO: 22 - protein, 131 aa, synthetic construct
  source 1..131: note = humanized VH-cl03H08
MGWSYIILFLVATATDGHSQVQLVQSGAEVKKPGSSVKLSCKASGYTFSSYWIHWVRQAPGQGLEWMGEINPSSGRTNYNEKFKNRVTITADKSTSTAYMELSNLRSEDSAVYYCASRGYWGQGTTLTVSS

SEQ ID NO: 23 - protein, 131 aa, synthetic construct
  source 1..131: note = humanized VL-cl04H08
METDTILLWVLLLWVPGSTGDIVLTQSPASLAVSLGERATINCKASQSVDYDGGSYMSWYQQRPGQPPKLLIYAASNLESGVPDRFSGSGSGTDFTLTIHSLQAEDVATYYCQQSYEDPLTFGGGTKVEIK

SEQ ID NO: 24 - protein, 131 aa, synthetic construct
  source 1..131: note = humanized VH-cl04H08
MGWSYIILFLVATATDGHSQVQLQQPGAEVKKPGSSVKLSCKASGYTFTSYWIHWVKQAPGQGLEWIGEINPSSGRTNYNEKFKNKVTITVDKSTSTAYMELSSLRSEDTAVYYCASRGYWGQGTTVTVSS

SEQ ID NO: 25 - DNA, 717 bp, synthetic construct
  source 1..717: mol_type = other DNA; note = humanized VL+CL-cl03E08 (nucleotides)
atggagacagacacaatcctgctatgggtgctgctgctctgggttccaggctccactggtgatattgtgctgacccagagcccggacagcctggcagtgagtctgggtcaacgcgccaccattaactgcaaagccagccagagcgtggattatgacggcggtagctacatgagctggtttcagcagaaaccgggtcagccgccgaaactgctgatttatgccgccagcaatctggaaagcggcgttccggcccgttttagcggtagcggcagcggcaccgattttaccctgaccattagctcgctgcaggcggaggatgtggccacctattactgccagcagagctacgaagatccgctgacctttggcggcggtaccaaagtggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 26 - DNA, 717 bp, synthetic construct
  source 1..717: mol_type = other DNA; note = humanized VL+CL-cl03G05 (nucleotides)
atggagacagacacaatcctgctatgggtgctgctgctctgggttccaggctccactggtgacatcgtgctgacccagtcccctgcttccctcgctgtgtccctgggcgagagggctaccatctcctgtaaggcctcccagtccgtggactacgacggcggctcctacatgtcctggttccagcagaggcctggccagcctcccaagctgctgatctacgctgcctccaacctggagtccggcgtgcctgataggttcagcggatccggctccggcaccgacttcaccctgaatatctcccctctggaggctgaggacgtggccgtgtactactgccagcagtcctacgaggaccctctgaccttcggcggcggcaccaaggtggagatcaagagaactgtcgcagcaccttccgtctttattttcccaccatcagatgaacagctgaaatccggtactgccagtgtggtctgtctgctcaataatttctatccccgggaggctaaagtccagtggaaagtggataatgcccttcagtccggcaactcccaggagagcgtgaccgagcaggattctaaggactcaacatattcactgtcatctactctcaccctgtctaaggctgattacgaaaagcacaaggtgtacgcttgcgaagttactcatcagggcctctcttcccctgtgaccaagagtttcaaccggggcgagtgctaa

SEQ ID NO: 27 - DNA, 717 bp, synthetic construct
  source 1..717: mol_type = other DNA; note = humanized VL+CL-cl03F04 (nucleotides)
atggagacagacacaatcctgctatgggtgctgctgctctgggttccaggctccactggtgacatcgtgatgacccagtcccctgactccctggctgtgtccctgggcgaaagggccaccatcaactgcaaggcctcccagtccgtggactacgacggcggctcctacatgtcctggtaccagcagaagcccggccagcctcccaagctgctgatctccgccgcttccaatctggagtccggcgtgcccgataggttttccggctccggctccggcaccgactttaccctgaccatccactccctgcaggccgaggacgtggctgtgtactactgccagcagtcctacgaggaccctctgaccttcggcgccggcaccaaggtggagatcaagagaactgtcgcagcaccttccgtctttattttcccaccatcagatgaacagctgaaatccggtactgccagtgtggtctgtctgctcaataatttctatccccgggaggctaaagtccagtggaaagtggataatgcccttcagtccggcaactcccaggagagcgtgaccgagcaggattctaaggactcaacatattcactgtcatctactctcaccctgtctaaggctgattacgaaaagcacaaggtgtacgcttgcgaagttactcatcagggcctctcttcccctgtgaccaagagtttcaaccggggcgagtgctaa

SEQ ID NO: 28 - DNA, 717 bp, synthetic construct
  source 1..717: mol_type = other DNA; note = humanized VL+CL-cl03H08 (nucleotides)
atggagacagacacaatcctgctatgggtgctgctgctctgggttccaggctccactggtgacatcgtgctgacccagtcccctgcttccctggctgtgtccctgggcgagagggctaccatctcctgcaaggcctcccagtccgtggactacgacggcggctcctacatgtcctggtaccagcagaagcccggccagcctcccaagctgctgatctacgccgcctccaacctggagtccggcgtgcccgataggttctccggatccggctccggcaccgacttcaccctgaacatctcctccgtgcaggaggaggacgtggccacctactactgccagcagtcctatgaggaccccctgaccttcggcgctggcaccaaggtggagatcaagagaactgtcgcagcaccttccgtctttattttcccaccatcagatgaacagctgaaatccggtactgccagtgtggtctgtctgctcaataatttctatccccgggaggctaaagtccagtggaaagtggataatgcccttcagtccggcaactcccaggagagcgtgaccgagcaggattctaaggactcaacatattcactgtcatctactctcaccctgtctaaggctgattacgaaaagcacaaggtgtacgcttgcgaagttactcatcagggcctctcttcccctgtgaccaagagtttcaaccggggcgagtgctaa

SEQ ID NO: 29 - DNA, 717 bp, synthetic construct
  source 1..717: mol_type = other DNA; note = humanized VL+CL-cl04H08 (nucleotides)
atggagacagacacaatcctgctatgggtgctgctgctctgggttccaggctccactggtgacatcgtgctgacccagtcccctgcttccctggctgtgtccctgggagagagggccaccatcaactgcaaggcctcccagtccgtggactacgacggcggctcctacatgtcctggtaccagcagaggcccggacagcctcccaagctgctgatctacgctgcctccaacctggagtccggcgtgcctgataggttttccggctccggctccggaaccgacttcaccctgaccatccactccctgcaggcggaggacgtggccacctactactgccagcagtcctacgaggaccccctgacattcggcggcggaaccaaggtggagatcaagagaactgtcgcagcaccttccgtctttattttcccaccatcagatgaacagctgaaatccggtactgccagtgtggtctgtctgctcaataatttctatccccgggaggctaaagtccagtggaaagtggataatgcccttcagtccggcaactcccaggagagcgtgaccgagcaggattctaaggactcaacatattcactgtcatctactctcaccctgtctaaggctgattacgaaaagcacaaggtgtacgcttgcgaagttactcatcagggcctctcttcccctgtgaccaagagtttcaaccggggcgagtgctaa

SEQ ID NO: 30 - DNA, 717 bp, synthetic construct
  source 1..717: mol_type = other DNA; note = chimeric VL(nucleotides)
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtggtagttatatgagttggttccaacagagaccaggacagccacccaaactcctcatctctgctgcatccaaccttgaatctggcatcccagccaggtttagtggcagtggctctgggacagacttcaccctcaatatccatcctgtggaggaggaggatgttgcaacctattactgtcagcaaagttatgaagacccgctcacgttcggtgctggtaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 31 - DNA, 1386 bp, synthetic construct
  source 1..1386: mol_type = other DNA; note = humanized VH+CH1+CH2+CH3-cl03E08, knob mutation, (nucleotides)
atgggatggagctatatcatcctctttttggtagcaacagctacagatggccactcccaggtgcagctgcagcagtcgggtgccgaagtgaaaaaaccgggtgctagtgtgaaactgagctgcaaagccagcggctataccttcaccagctactggatccactgggtgcgtcaggcgcctggccagggcctggaatggattggcgaaatcaatccgagcagcggtcgcaccaattacaatgaaaaatttaaaaatcgtgtgaccgttaccgtggataaaagcacctccaccgcctatatggagctgagcagcctgaccagcgaagatagcgccgtgtattattgcgccagccgcggttattggggccagggtaccaccctgaccgtgagcagcgctagcacgaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 32 - DNA, 1386 bp, synthetic construct
  source 1..1386: mol_type = other DNA; note = humanized VH+CH1+CH2+CH3-cl03G05, knob mutation, (nucleotides)
atgggatggagctatatcatcctctttttggtagcaacagctacagatggccactcccaggtgcagctggtgcagcccggcacagaggtgaagaagcctggcgcctccgtgaagctgtcctgcaaggcctccggctacaccttcaccagctactggatccactgggtgaggcaggctcctggacagggcctggagtggatgggcgagatcaacccctcctccggcaggaccaactacaacgagaagttcaagaaccgggtgaccatcaccgccgacaagtccacctccaccgcctacatgcagctgtcctccctgaggtccgaggacaccgccgtgtactactgcgcctccaggggatattggggccagggcaccacagtgaccgtgagctccgctagcacgaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 33 - DNA, 1386 bp, synthetic construct
  source 1..1386: mol_type = other DNA; note = humanized VH+CH1+CH2+CH3-cl03F04, knob mutation (nucleotides)
atgggatggagctatatcatcctctttttggtagcaacagctacagatggccactcccaggtgcagctgcagcagcccggagccgaagtgaagaagcccggctccagcgtgaagctgtcctgcaaggcctccggctacaccttcacctcctactggatccactgggtgaagcaggctcctggacagggcctggagtggatcggcgagatcaacccctcctccggccggaccaactacaacgagaagttcaagaacaaggtgaccatcaccgtggacaagtccacctccaccgcctacatggagctgtccaacctgcggtccgaggactccgccgtgtactactgtgcctccaggggctattggggccagggcacaaccctgaccgtgtcctccgctagcacgaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 34 - DNA, 1386 bp, synthetic construct
  source 1..1386: mol_type = other DNA; note = humanized VH+CH1+CH2+CH3-cl03H08, knob mutation, (nucleotides)
atgggatggagctatatcatcctctttttggtagcaacagctacagatggccactcccaggtgcagctggtgcagtccggcgccgaagtgaagaagcccggctcctccgtgaagctgagctgcaaggcctccggctacaccttctcctcctactggatccactgggtgaggcaggctcctggacagggcctggagtggatgggcgagatcaacccctcctccggcaggaccaactacaacgagaagttcaagaaccgggtgaccatcaccgccgacaagtccacctccaccgcctacatggagctgtccaacctgaggtccgaggactccgccgtgtactactgcgcctccaggggatattggggccagggcaccaccctgacagtgtcctccgctagcacgaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 35 - DNA, 1386 bp, synthetic construct
  source 1..1386: mol_type = other DNA; note = humanized VH+CH1+CH2+CH3-cl04H08, knob mutation, (nucleotides)
atgggatggagctatatcatcctctttttggtagcaacagctacagatggccactcccaggtgcagctgcagcagcctggagccgaggtgaagaagcccggctcctccgtgaagctgtcctgcaaggcctccggctacaccttcacctcctactggatccactgggtgaagcaggcccctggacagggcctggagtggatcggcgagatcaacccctcctccggcaggaccaactacaacgagaagttcaagaacaaggtgaccatcaccgtggacaagtccacctccaccgcctacatggagctgtcctccctgaggtccgaggacaccgccgtgtactactgtgcctccaggggctattggggccagggcacaaccgtgaccgtgtcctccgctagcacgaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 36 - DNA, 1386 bp, synthetic construct
  source 1..1386: mol_type = other DNA; note = chimeric VH+CH1+CH2+CH3, knob mutation, (nucleotides)
atgggatggagctatatcatcctctttttggtagcaacagctacagatggccactcccaggtccaactgcaacagcctgggactgaactggtgaagcctggggcttcagtgaagctgtcctgcaaggcttctggctacaccttcaccagttactggatacactgggtgaagcagaggcctggacaaggccttgagtggattggagagattaatcctagcagcggtcgtactaactacaacgagaaattcaagaacaaggtcacagtgactgtagacaaatcttccaccacagcctacatgcaactcagcaacctgacatctgaggactctgcggtctattactgtgcaagtaggggctactggggccaaggcaccactctcacagtctcctcagctagcacgaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 37 - DNA, 741 bp, synthetic construct
  source 1..741: mol_type = other DNA; note = human CH2+CH3, Hole mutations, (nucleotides)
atgggatggagctatatcatcctctttttggtagcaacagctacagatggccactccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagttgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 38 - DNA, 18 bp, synthetic construct
  source 1..18: mol_type = other DNA; note = His-TAG (nucleotide)
caccatcaccatcaccat

SEQ ID NO: 39 - protein, 6 aa, synthetic construct
  source 1..6: note = His-TAG
HHHHHH

SEQ ID NO: 40 - DNA, 108 bp, synthetic construct
  source 1..108: mol_type = other DNA; note = Strep-TAG (nucleotide)
ggtgccgcatggagccacccccagttcgaaaaaggggccgcatggagccacccccagttcgaaaaaggggccgcatggagccacccccagttcgaaaaaggggccgca

SEQ ID NO: 41 - protein, 36 aa, synthetic construct
  source 1..36: note = Strep-TAG
GAAWSHPQFEKGAAWSHPQFEKGAAWSHPQFEKGAA

SEQ ID NO: 42 - DNA, 39 bp, synthetic construct
  source 1..39: mol_type = other DNA; note = K842E sense nucleotide
gtacataatcctgtgtttgagccttttgaaaagccagtg

SEQ ID NO: 43 - DNA, 39 bp, synthetic construct
  source 1..39: mol_type = other DNA; note = K842E antisense nucleotide
cactggcttttcaaaaggctcaaacacaggattatgtac